(19) United States
(12) Patent Application Publication
(10) Pub. No.: US 2025/0312137 A1
(43) Pub. Date: Oct. 9, 2025

(54) AORTIC ARCH IMPLANTS AND RELATED SYSTEMS AND METHODS

(71) Applicant: inQB8 Medical Technologies, LLC, Burlington, MA (US)

(72) Inventors: Arshad Quadri, West Hartford, CT (US); J. Brent Ratz, Burlington, MA (US); Garrett Johnson, Costa Mesa, CA (US); Levi John Nicholas Caffes, Somerville, MA (US); Caroline Elizabeth Buckley, Belmont, MA (US); Jack Berkman Sattell, Cambridge, MA (US)

(73) Assignee: inQB8 Medical Technologies, LLC, Burlington, MA (US)

(21) Appl. No.: 19/170,286

(22) Filed: Apr. 4, 2025

Related U.S. Application Data
(60) Provisional application No. 63/575,171, filed on Apr. 5, 2024.
(60) Provisional application No. 63/575,351, filed on Apr. 5, 2024.
(60) Provisional application No. 63/712,363, filed on Oct. 25, 2024.

(51) Int. Cl. A61F 2/07

(52) U.S. Cl. CPC A61F 2/07; A61F 2002/072; A61F 2250/0065

(57) ABSTRACT

The present disclosure generally relates to prosthetic implants, prosthetic implant systems, and their methods of use, for example, for treating a dissection. The prosthetic implants contemplated herein comprise an expandable arch support structure comprising one or more expandable branches configured to be placed within one or more vessels. Certain aspects of the disclosure relate to methods for treating a dissection, e.g., an aortic dissection, using said prosthetic implants and implant systems. The methods comprise advancing one or more guidewires into an ascending aorta, a descending aorta, and/or one or more one or more vessels of an aortic root or aortic arch. The methods further comprise exposing one or more components of the expandable arch support structure from a sheath. In some cases, the exposed component is an expandable branch that is advanced into the one or more vessels of the aortic root or aortic arch, thus anchoring the implant to the native aorta.

### RELATED APPLICATIONS

This Application is a Non-Provisional of Provisional (35 USC 119 (e)) of U.S. Application Ser. No. 63/712,363, filed Oct. 25, 2024, entitled “ARCH GRAFT IMPLANTS AND RELATED METHODS” and a Non-Provisional of Provisional (35 USC 119 (e)) of U.S. Application Ser. No. 63/575,171, filed Apr. 5, 2024, entitled “ARCH GRAFT IMPLANTS AND RELATED METHODS” and a Non-Provisional of Provisional (35 USC 119 (e)) of U.S. Application Ser. No. 63/575,351, filed Apr. 5, 2024, entitled “AORTIC ARCH IMPLANTS AND RELATED SYSTEMS AND METHODS”. The entire contents of these applications are incorporated herein by reference in their entirety.

### FIELD

The present invention generally relates to implantable medical devices, and, more particularly, to prosthetic aortic implants, as well as systems and methods involving the same. Such devices, systems, and methods may be useful for e.g., the treatment of Acute Aortic Dissections (AAD), Intramural Hematomas and Thoracic Aortic Aneurysms.

### BACKGROUND

Management of AADs depend on the type of dissection and its location along the aorta, but generally involves medications, to reduce heart rate and lower blood pressure which help to prevent the ADD from worsening, and/or surgery, to remove as much of the dissected aorta as possible and to stop blood from leaking into the aortic wall. However, nearly 10-30% of all AADs are deemed inoperable and managed primarily with medication alone. The mortality in this population is high, with approximately 15-30% of patients dying within 24 hrs, which tapers off to approximately 1% per day from day 6 through day 30. Outcomes for surgical candidates are equally poor with sequela rates, e.g., mortality and neurological damage, as high as 15-30%. Accordingly, improved devices and methods are needed.

### SUMMARY

The present invention generally relates to implantable medical devices, and, more particularly, to a prosthetic aortic implant, systems comprising the prosthetic aortic implant, and related methods. The subject matter of the present disclosure involves, in some cases, interrelated products, alternative solutions to a particular problem, and/or a plurality of different uses of one or more systems and/or articles.

Aspects of the disclosure generally relate to a prosthetic implant comprising an expandable arch support structure comprising one or more openings and one or more expandable branches configured to be positioned within the one or more openings. In some embodiments, the expandable arch support structure is configured to be positioned within at least a portion of a descending portion of a native aorta. In some embodiments, the expandable arch support structure comprises a nonporous layer and is configured to contact an outer wall of the native aorta. In some embodiments, the expandable arch support structure comprises one or more changes in a cross-sectional dimension within the body of the expandable support structure. In some embodiments, the one or more expandable branches comprises a telescoping structure comprising a proximal portion, a middle portion, and a distal portion. In some embodiments, the one or more expandable branches is configured to be positioned within at least a portion of one or more vessels of an aortic arch of the native aorta and to permit blood flow from within the expandable arch support structure into at least one or more vessels of the aortic arch of the native aorta.

Some aspects of the present disclosure generally relate to prosthetic implant comprising a first expandable support structure interlocked to a second expandable support structure. For example, in some embodiments, the prosthetic implant comprise an expandable arch support structure, comprising one or more openings and one or more expandable branches configured to be positioned within the one or more openings, sized and configured to be positioned within a descending portion of a native aorta. In some embodiments, the prosthetic implant further comprises an expandable root support structure, comprising one or more openings and one or more expandable branches configured to be positioned within the one or more openings, sized and configured to be positioned within an ascending portion of a native aorta. In some embodiments, the distal end of the expandable root support structure is configured to engage a proximal end of the expandable arch support structure. In some embodiments, the expandable arch support structure comprises a second non-porous layer and is configured to contact an outer wall of at least a portion of the native aorta and/or one or more expandable branches comprising a telescoping structure. In some embodiments, the one or more expandable branches comprise a proximal portion, a middle portion, and a distal portion. In some embodiments, the one or more expandable branches is configured to be positioned within at least a portion of one or more vessels of an aortic arch of the native aorta and to permit blood flow from within the expandable arch support structure into one or more vessels of the aortic arch of the native aorta.

Aspects of the disclosure also relate to methods. In some embodiments, the method is a method of treating a dissection. In some embodiments, the methods comprise advancing a first guidewire into an ascending aorta and a descending aorta. In some embodiments, the methods comprise advancing a second guidewire into the ascending aorta and a left subclavian artery. In some embodiments, the methods comprise advancing a prosthetic implant delivery system into the ascending aorta over the first guidewire and the second guidewire. In some embodiments, the prosthetic implant delivery system comprises an outer sheath, an ascending sheath, and a descending sheath. In some embodiments, the ascending sheath carries an ascending portion of an expandable arch support structure. In some embodiments, the descending sheath carries a descending portion of the expandable arch support structure. In some embodiments, the descending sheath extends through the outer sheath. In some embodiments, the methods further comprise retracting the outer sheath in the ascending aorta to expose the descending sheath over the first guidewire. In some embodiments, the methods comprise advancing a first expandable branch of the expandable arch support structure from the outer sheath into the left subclavian artery over the second guidewire. In some embodiments, the expandable branch comprises a telescoping structure comprising a proximal portion, a middle portion, and a distal portion. In some embodiments, the methods further comprise advancing the descending sheath at least partially into the descending aorta, and exposing the ascending portion of the expandable arch support structure from the ascending sheath.

In some embodiments, the methods are directed to treating an aortic dissection. In some embodiments, the methods comprise advancing a first guidewire into a descending aorta and an ascending aorta. In some embodiments, the methods comprise advancing a second guidewire into the descending aorta and a brachiocephalic artery. In some embodiments, the methods comprise advancing a prosthetic implant system into a descending aorta over the first guidewire and the second guidewire. In some embodiments, the prosthetic implant delivery system comprises an outer sheath, an ascending sheath, and a descending sheath. In some embodiments, the ascending sheath carries an ascending portion of an expandable arch support structure, the descending sheath carrying a descending portion of the expandable arch support structure. In some embodiments, the descending sheath extends through the outer sheath. In some embodiments, the methods further comprise retracting an outer sheath in the descending aorta to expose the ascending sheath over the first guidewire. In some embodiments, the methods comprise advancing a first expandable branch of the second expandable support structure from the outer sheath into the brachiocephalic artery over the second guidewire. In some embodiments, the expandable branch comprises a telescoping structure comprising a proximal portion, a middle portion, and a distal portion. In some embodiments, the methods further comprise exposing the descending portion of the expandable arch support structure from the descending sheath.

In another set of embodiments, the methods for treating a dissection comprise advancing a first guidewire into an aorta. In some embodiments, the methods comprise advancing a second guidewire into the aorta and into at least a portion of a first vessel of an aortic arch. In some embodiments, the methods comprise advancing a prosthetic implant system into the aorta over the first guidewire and the second guidewire. In some embodiments, the prosthetic implant system comprises an outer sheath and an inner sheath extending through the outer sheath. In some embodiments, the inner sheath carries a second expandable support structure. In some embodiments, the methods further comprise retracting the outer sheath in the aorta to expose the inner sheath over the first guidewire. In some embodiments, the methods comprise advancing a first expandable branch of the expandable arch support structure from the outer sheath into the first head vessel over the second guidewire, wherein the expandable branch comprises a telescoping structure comprising a proximal portion, a middle portion, and a distal portion; and exposing the second expandable support structure portion from the inner sheath in the aorta.

Additional aspects of the disclosure relate to a graft delivery system, the system comprising an outer sheath. In some embodiments, the outer sheath is configured to move along a first guidewire and a second guidewire. In some embodiments, the graft delivery system further comprises an arch graft sheath inside the outer sheath. In some embodiments, the arch graft sheath is configured to move along a first guidewire. In some embodiments, the arch graft sheath is pre-loaded with an expandable support structure and configured to release the expandable arch support structure at least partially in a descending aorta and an ascending aorta. In some embodiments, the graft delivery system further comprises a first expandable branch of the expandable arch support structure pre-loaded in the outer sheath. In some embodiments, he first expandable branch is configured to move along the second guidewire.

In another set of embodiments, a graft delivery system comprises a distal capsule comprising a plurality of bearing elements, a distal locking ring and a proximal locking ring, and a prosthetic implant system comprising an expandable root support structure. In some embodiments, a proximal end of the expandable root support structure is connected to the distal locking ring. In some embodiments, a distal end of the expandable root support structure is connected to the proximal locking ring. In some embodiments, the plurality of bearing elements, the distal locking ring, and the proximal locking ring are configured to rotate about a guide wire lumen.

Other aspects of the disclosure relate to a prosthetic implant system. In some embodiments, the prosthetic implant system comprises an outer sheath. In some embodiments, the outer sheath is configured to move along a first guidewire and a second guidewire. In some embodiments, the prosthetic implant system comprises an inner sheath inside the outer sheath. In some embodiments, the inner sheath is configured to move along a first guidewire. In some embodiments, the inner sheath is configured to carry an expandable arch support structure. In some embodiments, the prosthetic implant system further comprises a first expandable branch of the expandable arch support structure inside the outer sheath. In some embodiments, the first expandable branch is configured to move along the second guidewire.

In another aspect, the present disclosure generally encompasses methods of making one or more of the embodiments described herein, for example, prosthetic aortic implant. In still another aspect, the present disclosure encompasses methods of using one or more of the embodiments described herein, for example, prosthetic aortic implant.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 shows an exemplary expandable arch support structure comprising a first expandable branch for placement into a left subclavian artery a second expandable branch for placement into the left common carotid artery, and a third expandable branch for placement into a brachiocephalic artery. In some embodiments, the exemplary expandable arch support structure further comprises a coupling at an ascending portion for anchoring to an expandable root support structure.

FIGS. 2A-2B shows an exemplary embodiment of an expandable branch of an expandable arch support structure as contemplated herein. In some embodiments, the expandable branch comprises a proximal portion, a middle portion, and a distal portion. In some embodiments, the proximal portion comprises a layer such as ePTFE. In some embodiments, the second portion comprises ePTFE and an expandable support structure. In some embodiments, the third portion comprises an expandable support structure.

FIG. 3 shows an exemplary embodiment of an expandable arch support structure interlocked to an expandable arch root support structure. In some embodiments, one or more expandable branches of the expandable arch support structure are configured to be placed in a brachiocephalic artery, a left common carotid artery, and/or a subclavian artery. In some embodiments, the one or more expandable branches of the expandable root support structure are configured to be placed in a left coronary artery and/or a right coronary artery.

FIGS. 4A-4G shows the transapical delivery of an exemplary prosthetic implant delivery system as contemplated herein, according to one set of embodiments. The prosthetic implant delivery system is guided into the ascending aorta via a first and a second guide wire (FIG. 4A). The first guide wire is placed in a descending aorta and the second guidewire is placed into a left subclavian artery (FIG. 4A). After advancing the prosthetic implant delivery system into the ascending aorta, the outer sheath is retracted proximally to expose the ascending sheath, nosecone and the descending sheath (FIG. 4B). Preplaced guidewires are then advanced to access the left common carotid artery and the brachiocephalic artery, respectively (FIG. 4C). An expandable branch of an expandable arch support graft is advanced over the preloaded guide wires. As described elsewhere herein, this is achieved via a telescoping feature built into the expandable branch structure (FIG. 4D). The implant is further advanced into the left subclavian artery/descending aorta ostium (FIG. 4E). The descending sheath and nosecone are pushed distally to deploy the descending portion of the expandable arch support graft, and the three expandable branches are balloon expanded within the head vessels of the aortic arch (FIG. 4F). The ascending sheath is retracted to deploy the ascending portion of the graft (FIG. 4G).

FIGS. 5A-5E illustrate an exemplary set of embodiments wherein a prosthetic implant delivery system is delivered transfemorally, according to some embodiments.

FIGS. 6A and 6B illustrate an exemplary prosthetic implant delivery system configured to permit independent rotation of an exemplary prosthetic implant loaded within the delivery system (from top to bottom).

FIG. 7 is a computer automated drawing illustrating the assembly of the nosecone and the distal locking ring.

FIG. 8 illustrates the connection between the distal locking ring and the proximal end of an exemplary implant.

FIGS. 9A and 9B illustrates transfemoral delivery of a prosthetic implant using the prosthetic implant delivery system disclosed herein. FIG. 9A shows a coronary access wire in a first position. FIG. 9B shows that the coronary access wire has been rotated by 180 degrees. Rotation is accomplished via knob on the handle of the delivery device (see FIG. 5A for location of rotation knob on device).

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

The present disclosure generally relates to implantable medical devices, and, in some embodiments, to a prosthetic implant. Such implantable devices may be useful in the treatment of acute aortic dissections (AADs). In some embodiments, the prosthetic implant comprises an expandable support structure. The expandable support structure may comprise one or more expandable branches. The expandable branch may comprise a proximal portion, a middle portion, and a distal portion configured to permit flattening, telescoping, pivoting, and/or expansion of the one or more portions of the expandable branch. The one or more expandable branches may be configured to be placed into a vessel, e.g., coronary vessel or aortic arch head vessel. Additionally, in some cases a first expandable support structure comprising one or more expandable branches may be coupled to a second expandable support structure comprising one or more expandable branches.

The prosthetic implants may be implanted (e.g., surgically) in a subject to treat a disease, disorder, or other clinically recognized condition, or for prophylactic purposes, and/or may have a clinically significant effect on the body of the subject to treat and/or prevent the disease, disorder, or condition. A “subject” refers to any animal such as a mammal (e.g., a human). Non-limiting examples of suitable subjects include a human, a non-human primate, a cow, a horse, a pig, a sheep, a goat, a dog, a cat or a rodent such as a mouse, a rat, a hamster, a bird, a fish, or a guinea pig. Generally, the invention is directed toward use with humans. In some embodiments, a subject may demonstrate health benefits, e.g., upon implantation of the prosthetic aortic implant.

In some embodiments, the prosthetic devices disclosed herein are useful for the treatment of subjects suffering from one or more types of Acute Aortic Dissections (AADs). As would be understood by those of ordinary skill in the art, AADs generally occur when a portion of the aortic intima (the inner most layer of the aorta) ruptures and systemic blood pressure serves to delaminate the intimal layer from the media layer resulting in a false lumen for blood flow that can propagate in multiple directions along the length of the aorta. AADs that occur in the ascending portion of the aorta may generally be classified as Acute Type A Aortic Dissections (ATAADs, also referred to as Type 1 and Type 2 according to De Bakey classification system), whereas those not involving the ascending aorta are referred to as Type B dissections (according to the Stanford classification system). In some cases, failure to rapidly treat AADs, and particularly, ATAADs, may lead to severe sequela including stroke, organ damage, e.g., kidney failure or life-threatening intestinal damage, aortic valve damage, and death due to severe internal bleed (e.g., mortality rate is nearly 50% at 48 hours post injury and 90% within 30 days post injury).

Those of ordinary skill will understand, based upon the teachings of this specification that the systems, methods, and devices described herein may, in some embodiments, fill an important therapeutic gap in the treatment of patients with AADs. For example, the prosthetic aortic implants described herein may advantageously be useful for providing a prophylactic that may be administered non-invasively in an outpatient setting. In some embodiments, the prosthetic aortic implants described herein may advantageously be administered to patients with recently diagnosed aortic aneurysms, for example, as a preventative measure to delay (or prevent) disease progression. In other embodiments, the prosthetic aortic implants may advantageously be useful for rapidly treating patients suffering from ATAADs (e.g., an aortic dissection in the ascending aorta that occur acutely and rapidly without warning, as may occur in patients with undiagnosed aortic aneurysms). In some embodiments, placement (e.g., implantation) of the prosthetic aortic implant within the ascending aorta of a patient suffering from ATAAD may serve to reinforce the inner wall of the aorta near the dissection and re-establish a true lumen for blood to flow through. In some embodiments, the prosthetic aortic implants described herein may advantageously provide a non-invasive method to fix damaged aortic valves, for example, by incorporating a valve frame configured to reversibly (or irreversibly) receive a prosthetic aortic valve. For example, in some embodiments, the prosthetic aortic implants described herein may be sized and configured to receive (e.g., reversibly) a transcatheter aortic valve implant (TAVI). In some embodiments, an aortic valve such as a TAVI is positioned within the prosthetic aortic implant and/or a portion of the native aorta that has been configured to receive the TAVI e.g., as a result of the presence of the prosthetic aortic implant.

The prosthetic aortic implants described herein may have several advantages over previously described devices. For example, some previously described devices generally comprise a short one-piece implant constructed of fabric with built-in reinforcements configured to reside within the ascending aorta alone. However, such devices may be prone to movement and dislocation e.g., because they generally lack features that may anchor the device to the native aorta. In contrast to traditional devices, the prosthetic implants described herein comprise, in some embodiments, one or more expandable anchoring structures, configured to engage and apply a radial outward force, to one or more structures of a native aorta, e.g., aortic sinuses and/or the sinotubular junction, within an aortic root of the native aorta, thus anchoring the device to the native aorta (e.g., reducing the likelihood of movement and/or dislocation). In some embodiments, the disclosed devices are configured to extend from the ascending aorta into the descending aorta, wherein the descending portion further anchors the device to the native aorta, e.g., advantageously further reducing the likelihood of movement and/or dislocation. Additionally, in some embodiments, the prosthetic implants described herein comprise one or more expandable branches, configured to engage and apply a radial outward force, to one or more structures of a native aorta, e.g., a right and/or left coronary artery.

In some cases, aortic grafts for treating aortic aneurysms may be used to treat ATAADs, wherein the aortic grafts generally comprise a non-porous layer to wall off the aneurysm from the main lumen of the graft and aorta. However, such grafts cannot generally be placed over regions of the aorta (e.g., the aortic arch) e.g., that require fenestration windows so blood may flow to branched vessels. The prosthetic implants described herein may advantageously comprise, in some embodiments, one or more expandable branches configured to sit within a branched vessel (e.g., brachiocephalic artery, left common carotid artery, left subclavian artery, left and/or right coronary arteries). Additionally, in alternative embodiments, the prosthetic implants described herein may comprise a porous layer positioned over at least part of an expandable support structure, e.g., thereby permitting the graft to span from the ascending aorta into the descending aorta without blocking blood flow to critical branch vessels (e.g., brachiocephalic artery, left common carotid artery, and the left subclavian artery).

In some cases, bare-metal implants have also been described for the treatment of AADs. However, bare-metal frames are generally abrasive and may erode through the tissue and/or cause the fragile intima layer to dissect further. The prosthetic implants described herein may advantageously comprise, in some embodiments, an expandable reinforcement structure comprising an atraumatic outer layer configured to distribute a radial force throughout the entire aorta, e.g., thereby reducing the likelihood of the aneurysms rupturing.

In some embodiments, a prosthetic implant comprises an expandable support structure. In some embodiments, the expandable support structure is capable of being expanded from a crimped state to an expanded state. In some embodiments, the expandable support structure is configured to be placed within an aortic arch of a native aorta, and is herein generally referred to as an expandable arch support structure. In some embodiments, the expandable support structure is configured to be placed within an aortic root of a native aorta, and is herein generally referred to as an expandable root support structure. In some embodiments, the expandable support structure comprises one or more expandable branches. In some embodiments, the one or more expandable branches comprises a proximal portion, a middle portion, and a distal portion. In some embodiments, a proximal portion comprises an expandable support structure. In some embodiments, the middle portion comprise an expandable support structure. In some embodiments, the distal portion comprises an expandable support structure.

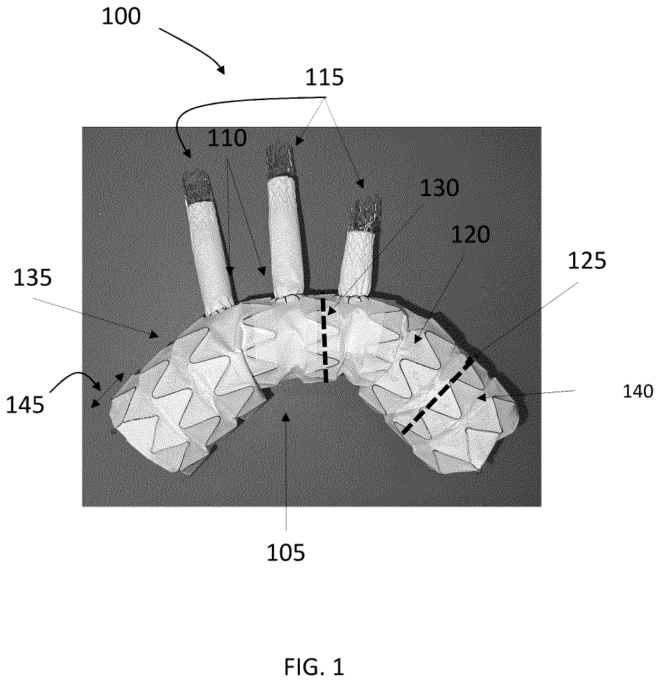

In some embodiments, a prosthetic implant is configured to be positioned within an aortic arch of a subject in need thereof. FIG. 1 shows exemplary aortic arch implant 100 comprising an expandable arch support structure 105 comprising one or more openings 110 and one or more expandable branches 115 configured to be positioned within the one or more openings 110 (FIG. 1). In some embodiments, an ascending portion 135 of expandable arch support structure 105 is configured to be positioned within at least a portion of an ascending portion of a native aorta. descending portion of a native aorta. In some embodiments, a descending portion 140 of expandable arch support structure 105 is configured to be position within at least a portion of a descending portion of a native aorta. In some embodiments, the expandable arch support structure comprises a nonporous layer 120 configured to contact an outer wall of the native aorta. In some embodiments, the nonporous layer 120 comprises ePTFE. In some embodiments, the expandable arch support structure 105 comprises one or more changes in a cross-sectional dimension within the body of the expandable arch support structure. For example, in some cases, a distal portion and a proximal portion of the expandable arch support structure have a first cross-sectional dimension 125 different (e.g., larger) than a middle portion of the expandable arch support structure (e.g., second cross-sectional dimension 130). In some embodiments, expandable arch support structure 105 further comprises a coupler 145, for example, for coupling to a second expandable support structure, such as an aortic root support structure as disclosed in International Patent Application entitled, “Aortic Root Implants And Related Systems And Method,” filed on the same day herewith, the entire contents of which is incorporated herein by reference in its entirety.

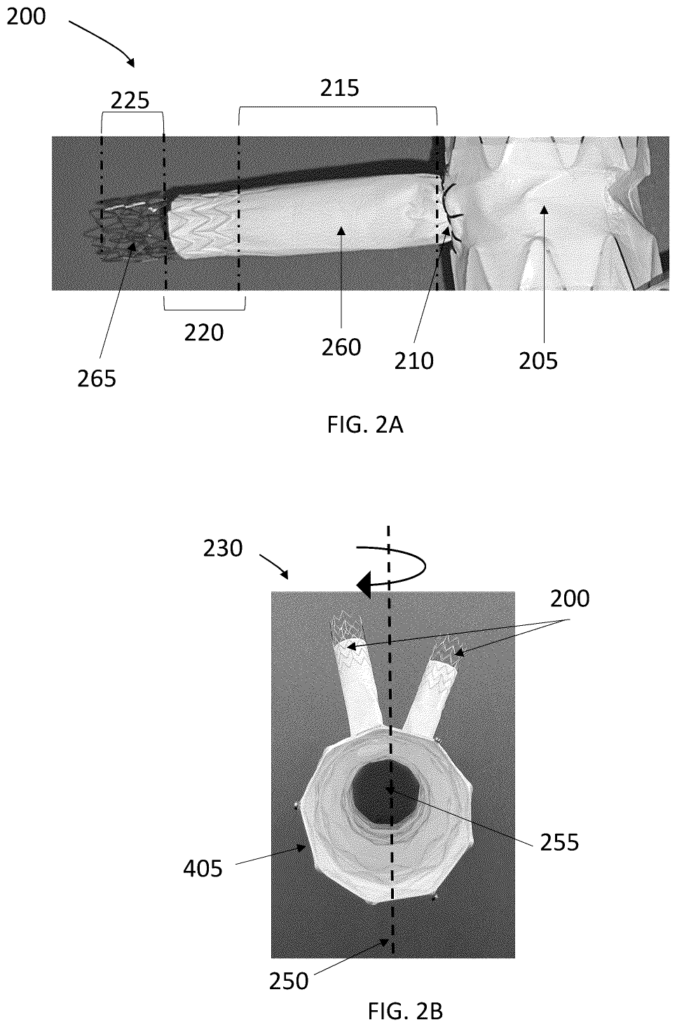

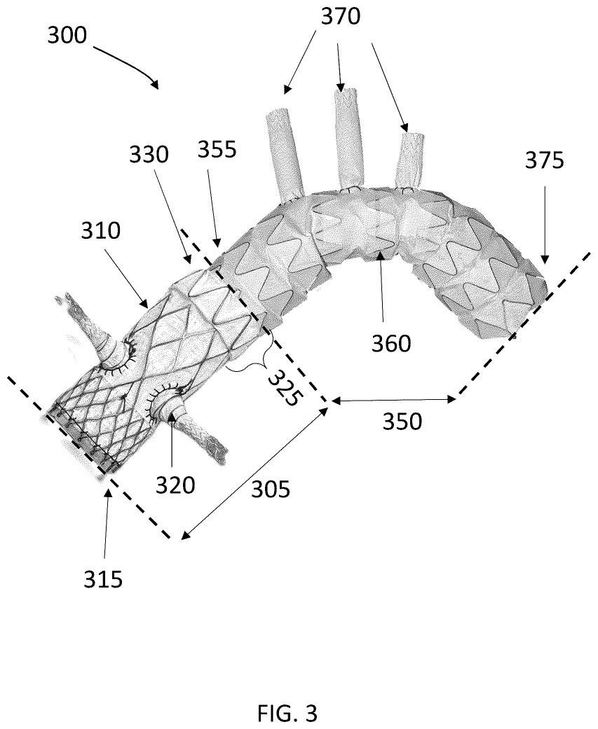

FIG. 2 illustrates an exemplary expandable branch 200 positioned within opening 210 of expandable arch support structure 205. In some embodiments, exemplary expandable branch 200 of expandable arch support structure 205 comprises a telescoping structure comprising a proximal portion 215, a middle portion 220, and a distal portion 225. As used herein, the term “telescoping structure” refers to any structure that permits the reversible extension and collapse of the expandable branch. Exemplary embodiments of telescoping structures are shown in FIGS. 2 and 3. For example, in some embodiments, the telescoping structure comprises an ePTFE structure (e.g., proximal portion in FIG. 2). In other embodiments, the telescoping structure comprises a metallic wire frame comprising a concentric cone geometry (e.g., expandable branch of expandable root support structure for placement into a coronary artery). Both structures permit collapse of the distal portion 225 and the middle portion 220 into the proximal portion 215 of the exemplary expandable branch 200. In some cases, the distal portion 225 and/or middle portion 220 are at least partially collapsed into a lumen of the expandable arch support structure 205.

FIG. 2B shows an exemplary expandable arch support structure 230 comprising expandable arch support structure 205. In some embodiments, the one or more expandable branches 200 are offset from each other relative to first axis 250. In some embodiments, one or more expandable branches 200 is tubular and comprises a central lumen 255 that extends from a proximal portion 215 to a distal portion 225 of the one or more expandable branches 200. In some embodiments, the central lumen 255 of the one or more expandable branches 200 is in fluidic communication with the central lumen 255 of the expandable arch support structure 205.

As stated above, FIG. 2A illustrates a telescoping structure of the one or more expandable branches 200, according to some embodiments. In some embodiments, the one or more branches 200 comprises proximal portion 215, middle portion 220, and distal portion 225. As shown in FIG. 2A, in some embodiments, proximal portion 215 comprises a telescoping frame comprising nonporous layer 260 having a tubular and/or cylindrical structure. However, this embodiment is not limiting, and nonporous layer 260 may be, in some cases, configured into any suitable geometry known to the skilled artisan for use as contemplated herein. In some embodiments, proximal portion 215 does not contain a metal frame therewith (e.g., an expandable frame is not present with nonporous layer 260). In some embodiments, such configurations are useful, for example, for folding expandable branch 200 into a configuration parallel to the expandable arch support structure 205 and/or inverting the expandable branch 200 into the central lumen 255 of the expandable arch support structure 205 (e.g., for delivery and/or deployment of said implant). Those of skill in the art will understand, however, that the figures are not meant to be limiting in any way, and that proximal portion 215 may comprise a metallic frame (e.g., a telescoping metal frame or expandable frame) in some embodiments. In some embodiments, proximal portion 215 may comprise a frame (e.g., metallic frame) and nonporous layer 260.

In some embodiments, one or more branches 200 comprises a distal portion 225 comprising a distal expandable frame 265. In some embodiments, distal expandable frame 265 is configured to expand from a crimped/closed position into an expanded position within at least one of the head vessels of the aortic arch (e.g., brachiocephalic artery, left subclavian artery, and/or the left common carotid artery). In some embodiments, expansion from a crimped/closed position into an expanded position anchors the implant to the head vessel.

In some embodiments, middle portion 220 comprises a portion of distal expandable frame 265 and nonporous layer 260. In some embodiments, nonporous layer 260 is provided over, within, or interwoven into distal expandable frame 265 within middle portion 220 of the one or more expandable branches 200. Those of skill in the art will understand and appreciate that upon expansion, at least a portion of distal expandable frame 265, middle portion 220 radially compresses against the intima of the head vessel (e.g., pressing nonporous layer 260 against interior wall of head vessel). In some embodiments, such configurations are useful, for example, for preventing leakage of body fluid (e.g., blood) from the lumen of expandable branch after placement at a target location (e.g., brachiocephalic artery, left subclavian artery, and/or the left common carotid artery).

In some embodiments, one or more expandable branches of an expandable arch support structure is configured to be positioned within at least a portion of one or more vessels of an aortic arch of the native aorta. It is believed that such configurations better permit blood flow from within the expandable arch support structure into the vessels of the aortic arch of the native aorta (e.g., brachiocephalic artery, left common carotid artery, and left subclavian artery). Additionally, it is believed that upon expanding the expandable branch within the vessel of the aortic arch anchors the implant to the native aorta.

In some embodiments, a prosthetic implant is configured to be positioned within an aortic root and an aortic arch of a subject in need thereof. In some embodiments, a prosthetic implant comprises an expandable arch support structure interlocked with an expandable root support structure. In some embodiments, the expandable arch support structure comprising one or more openings, and one or more expandable branches configured to be positioned within the one or more openings is sized and configured to be positioned within the descending portion of the native aorta. In some embodiments, the expandable arch support structure comprises a second nonporous layer and is configured to contact an outer wall of at least a portion of a native aorta. In some embodiments, the expandable arch support structure comprises one or more expandable branches comprising the telescoping structure, and optionally, and a tapered geometry. In some embodiments, the one or more expandable branches comprises a proximal portion, a middle portion, and a distal portion. In some embodiments, the one or more expandable branches is configured to be positioned within at least a portion of one or more vessels of an aortic arch of the native aorta and to permit blood flow from within the expandable arch support structure into one or more vessels of the arch of the native aorta.

As described above, in some embodiments, the expandable arch support structure is interlocked with an expandable root support structure. Any suitable method of interlocking the two support structures known in the art may be used by the skilled artisan. In some embodiments, the distal end of the expandable root structure is configured to engage a proximal end of the expandable arch support structure. In some embodiments, an expandable root support structure, comprising one or more openings, and one or more expandable branches configured to be positioned within the one or more openings, is sized and configured to be positioned within an ascending portion of a native aorta.

In some embodiments, an expandable root support structure comprises a first non-porous layer (e.g., ePTFE) and is configured to contact an outer wall of the native aorta. In some embodiments, the expandable root support structure comprises one or more expandable branches comprising a telescoping structure and/or a tapered geometry. In some embodiments, the one or more expandable branches is configured to be positioned within at least a portion of at least one coronary artery of an aortic root of the native aorta. It is believed that, upon implantation in a subject, such configuration permit blood flow from within the expandable root support structure into at least one coronary artery of the aortic root of the native aorta.

Additionally, in some embodiments, a proximal end of an expandable root support structure comprises a valved conduit. In some embodiments, the valved conduit comprises a bovine aortic valve. In some embodiments, the valved conduit comprises a porcine tissue valve. In some embodiments, the valved conduit at the proximal end of the expandable root support structure is removable. In some embodiments, the proximal end of the expandable root support structure comprises a prosthetic aortic valve frame. In some embodiments, the prosthetic aortic valve frame comprises a bridge valve. In some embodiments, the prosthetic aortic valve frame comprises a destination valve. In some embodiments, the prosthetic aortic valve frame is configured to receive the aortic valve implant. In some embodiments, the prosthetic aortic valve frame does not comprise a valve. In some embodiments, the aortic valve frame reversibly receives the aortic valve implant. In some embodiments, the aortic valve frame irreversibly receives the aortic valve implant. In some embodiments, a proximal end of the prosthetic aortic valve frame is flared and extends into a left ventricular outflow track (LVOT), thereby at least partially anchoring the prosthetic implant to the native aorta.

In some embodiments, the proximal end of the expandable root support structure is in contact with but not directly adhered and/or grafted to the native aorta upon deployment in the native aorta.

FIG. 3 shows exemplary modular prosthetic implant 300 as contemplated herein. In some embodiments, a distal end 330 of aortic root implant 305 is coupled to a proximal end 355 of aortic arch implant 350 via coupling structure 325. As described elsewhere herein, aortic root implant 305, comprises expandable root support structure 310 and one or more expandable branches 320, which is configured to sit within an aortic root and the coronary ostia, respectively, of a subject. In some embodiments, the one or more expandable branches 320, upon expansion within the coronary ostia, is configured to apply a radial force to the coronary arteries, thus anchoring said implant to the aortic root. Additionally, in some embodiments, expandable root support structure may further comprise an expandable anchoring structure at proximal end 315. In some embodiments, the expandable anchoring structure may be configured to be positioned within the aortic root of a subject and apply radial force to one or more of the sinuses of the aortic root and/or the sinotubular junction when expanded.

In some embodiments, aortic arch implant 350 comprises expandable arch support structure 360 configured to extend from the descending aorta, through the aortic arch and into the ascending aorta of a subject. In some embodiments, aortic arch implant 350 comprises one or more expandable branches 370 configured to sit within at least one head vessel of the aortic arch (e.g., brachiocephalic artery, left subclavian artery, and/or left common carotid artery). In some embodiments, the one or more expandable branches 370, upon expansion within the head vessels, is configured to apply a radial force to the intima of the head vessels, thus anchoring said implant to the aortic arch of a native aorta. Additionally, a distal end 375 of expandable arch support structure 360, upon expansion within at least a portion of the descending aorta is configured to apply a radial force to the intima of the descending aorta further anchoring the implant to the native aorta.

Without wishing to be bound by any particular theory, it is believed that interlocking an expandable arch support structure to an expandable root support structure permits multiple anchoring points of the prosthetic implant to the native aorta without exerting excessive force on the structures within the native aorta.

In some embodiments, a prosthetic implant, as contemplated herein, comprises an expandable branch of an expandable arch support structure. In some embodiments, the proximal portion of the one or more expandable branches does not comprise an expandable support structure (e.g., an expandable metallic wire frame). In some embodiments, the proximal portion of the one or more expandable branches comprises a nonporous layer (e.g., ePTFE). In some embodiments, the nonporous layer is the ePTFE. In some embodiments, the middle portion of the one or more expandable branches comprises an expandable support structure (e.g., an expandable metallic wire frame). In some embodiments, the expandable branch support structure is at least partially covered by a nonporous layer (e.g., ePTFE). In some embodiments, the expandable branch support structure is at least partially covered by an ePTFE layer. In some embodiments, the distal portion of the one or more expandable branches comprises an expandable support structure (e.g., an expandable metallic wire frame). In some embodiments, the distal portion of the one or more expandable branches comprising the expandable support structure is not covered in a non-porous layer (e.g., ePTFE). In some embodiments, the distal portion of the one or more expandable branches comprising the expandable support structure is not covered in ePTFE.

In some embodiments, one or more expandable branches of an expandable arch support structure is configured to collapse within at least a portion of the implant. It is believed that such configurations are advantageous, for example, for delivering the implant to the desired anatomical location. For example, in some embodiments, when a middle and distal portions comprises an expandable support structure and the proximal portion comprises an ePTFE telescoping structure and when the middle portion and the distal portion of the one or more expandable branches are in a crimped state, the middle portion and distal portions are configured to collapse into the proximal portion of the one or more expandable branches. In some embodiments, the proximal portion, middle portion, and the distal portions may also collapse, at least partially, into a lumen of the expandable arch support structure.

In some embodiments, a prosthetic implant, as contemplated herein, comprises one or more expandable branches of an expandable root support structure. In some embodiments, the one or more expandable branches are configured to be placed within a coronary artery (e.g., right and/or left coronary artery). Those of skill in the art will understand and appreciate that the anatomical features of the coronaries may be different, or the same, as the vessels in the aortic arch. Thus, in some embodiments, the one or more expandable branches of the expandable root support structure have a different geometry and/or configuration than the one or more branches of an expandable arch support structure. For example, in some embodiments, one or more expandable branches of the expandable root support structure comprise a proximal portion, a middle portion, and a distal portion. In some embodiments, the proximal portion of the one or more expandable branches of the expandable root support structure does not comprise a metallic wire frame. In some embodiments, the middle portion of the one or more expandable branches of the expandable root support structure comprises a metallic wire frame. The metallic wire frame, in some embodiments, comprises an ePTFE covering. In some embodiments, the distal portion of the one or more expandable branches of the expandable root support structure comprises an expandable support structure (e.g., an expandable metallic wire frame). In some embodiments, expandable support structure of the distal portion comprises an ePTFE covering, optionally, wherein the ePTFE covering, comprises one or more pores. In some embodiments, when the expandable support structure of the distal portion is in a crimped geometry. The one or more pores in the ePTFE covering are open (e.g., patent). In some embodiments, when the expandable support structure of the distal portion is an expanded geometry. The one or more pores in the ePTFE covering are closed. It is believed that such configurations may be useful, for example, to preserve coronary blood flow during anchoring of the expandable root support structure to the coronary arteries.

As with the expandable arch support structure, one or more expandable branches of an expandable root support structure are also configured to collapse within at least a portion of the implant. Again, it is believed that such configurations are advantageous, for example, for delivering the implant to the desired anatomical location. Thus, in some embodiments, when a metallic wire frame of a middle portion of the one or more expandable branches is in a collapsed state, the middle portion is configured to sit within a proximal portion of the one or more expandable branches (e.g., the proximal portion does not comprise a metallic wire frame). In some embodiments, when an expandable support structure of a distal portion of the one or more branches is in a crimped state and the middle portion is in a collapsed state, the distal and middle portions are configured to fit within the proximal portion of the expandable branch and/or within a lumen of the expandable root support structure.

In some embodiments, one or more expandable branches of an expandable arch support structure and/or an expandable root support structure are configured to have similar, or different, structures and/or configurations. For example, in some embodiments, a proximal portion of the one or more expandable branches is configured to be coupled to the middle portion of the one or more expandable branches; and the middle portion of the one or more expandable branches is configured to be coupled to the distal portion of the one or more expandable branches.

In some embodiments, one or more expandable branches of an expandable arch support structure and/or an expandable root support structure are configured to pivot radially between 0 degrees and 360 degrees, relative to a first axis. In some embodiments, the first axis runs parallel to an elongate central passageway defined by the one or more expandable branches. In other embodiments, one or more expandable branches of an expandable arch support structure and/or an expandable root support structure are configured to pivot laterally between 90 degrees and −90 degrees, relative to a second axis. In some embodiments, the second axis runs perpendicular to the elongate central passageway defined by the one or more expandable branches.

In some embodiments, one or more expandable branches of an expandable arch support structure and/or an expandable root support structure are configured to be in an extended state. In other embodiments, the one or more expandable branches of an expandable arch support structure and/or an expandable root support structure are configured to be in a collapsed state.

In some embodiments, one or more expandable branches of an expandable arch support structure and/or an expandable root support structure are configured to have a conical geometry. In some embodiments, in an expanded state, an inner diameter of the proximal portion of the one or more expandable branches is larger than an inner diameter of the distal portion of the one or more expandable branches.

In some embodiments, one or more expandable branches of an expandable arch support structure and/or an expandable root support structure is configured to have a cylindrical geometry. In some embodiments, in an expanded state, an inner diameter of the proximal portion of the one or more expandable branches is approximately the same as an inner diameter of the distal portion of the one or more expandable branches.

In some embodiments, one or more expandable branches of an expandable arch support structure and/or an expandable root support structure comprise a metallic wire frame (e.g., a proximal portion, a middle portion, or a distal portion) and/or expandable support structure (e.g., a proximal portion, a middle portion, or a distal portion) covered by a nonporous layer (e.g., ePTFE). In some embodiments, the nonporous layer comprises ePTFE.

In some embodiments, one or more expandable branches of an expandable arch support structure and/or an expandable root support structure comprise a metallic wire frame (e.g., a proximal portion, a middle portion, or a distal portion) and/or expandable support structure (e.g., a proximal portion, a middle portion, or a distal portion) covered by a porous layer In some embodiments, the porous layer comprises ePTFE.

As described above, a primary function of the one or more expandable branches of an expandable arch support structure is to anchor to the vessels of the aortic arch and to permit blood flow from within the expandable arch support structure into at least the left subclavian artery, the left common carotid artery, and/or the brachiocephalic artery of the native aorta. Thus, in some embodiments, the expandable arch support structure comprises a first expandable branch configured to be positioned within at least a portion of the left subclavian artery. In some embodiments, the expandable arch support structure comprises a first expandable branch configured to be positioned within at least a portion of the left common carotid artery. In some embodiments, the expandable arch support structure comprises a first expandable branch configured to be positioned within at least a portion of the brachiocephalic artery. In some embodiments, the expandable arch support structure comprises a first expandable branch configured to be positioned within at least a portion of the left common carotid artery and a second expandable branch configured to be positioned within at least a portion of the brachiocephalic artery. In some embodiments, the expandable arch support structure comprises a first expandable branch configured to be positioned within at least a portion of the left common carotid artery and a second expandable branch configured to be positioned within at least a portion of the subclavian artery. In some embodiments, the expandable arch support structure comprises a first expandable branch configured to be positioned within at least a portion of the brachiocephalic artery and the second expandable branch configured to be positioned within at least a portion of the subclavian artery. In some embodiments, the expandable arch support structure comprises a first expandable branch configured to be positioned within at least a portion of the brachiocephalic artery, a second expandable branch is configured to be positioned within at least a portion of the left common carotid artery, and a third expandable branch is configured to be positioned within at least a portion of the subclavian artery.

Likewise, a primary function of the one or more expandable branches of an expandable root support structure is to anchor to the coronary arteries and to permit blood flow from within the expandable root support structure into a left coronary artery and/or right coronary artery. Thus, in some embodiments, the expandable root support structure comprises a first expandable branch configured to be positioned within at least a portion of a left coronary artery. In some embodiments, the expandable root support structure comprises a first expandable branch configured to be positioned within at least a portion of a right coronary artery. In some embodiments, the expandable root support structure comprises a first expandable branch configured to be positioned within at least a portion of the left coronary artery and the second expandable branch is configured to be positioned within at least a portion of the right coronary artery. In some embodiments, the distal portion of the first expandable branch and the distal portion of the second expandable branch each comprise an expandable support structure, that when expanded, is configured to apply a radially outward force to a coronary artery, thereby anchoring the implant to the native aorta. In some embodiments, the first expandable branch and the second expandable branch of the expandable root support structure is configured to keep open a right and/or left coronary ostium. In some embodiments, the expandable root support structure comprises a first expandable branch positioned e.g., 120 degrees (e.g., from 90 degrees to 170 degrees) from a second expandable branch, relative to a second axis. In some embodiments, the first expandable branch and/or second expandable branch are positioned at least 15 mm (e.g., at least 5-25 mm) above the proximal end of the expandable root support structure.

In an alternative set of embodiments, an expandable root support structure is anchored to the aortic root using an expandable anchoring structure (e.g., as opposed to anchoring to the coronary vessels). In some embodiments, the expandable anchoring structure comprises an expandable trilobe structure. In some embodiments, at least a portion of the expandable trilobe structure applies a radially outward force to an aortic sinus thereby anchoring the prosthetic implant to the native aorta. In some embodiments, the expandable trilobe structure further comprises a trilobe structure comprising three lobes, wherein each of the three lobes is sized and configured to conform to a curvature of the aortic sinus when the expanded anchoring structure is expanded. In some embodiments, each of the three lobes comprises one or more apex at a distal end of the expandable trilobe structure configured to be positioned adjacent to an aortic valve annulus of the patient. In some embodiments, the trilobe structure is configured to expand to engage an inner wall of the aortic sinus separately from expansion of the expandable root support structure, and the prosthetic implant is configured to sequentially deploy the trilobe structure before the expandable root support structure. In some embodiments, the one or more native leaflets are secured between the proximal end of the expandable root support structure and the one or more backstop elements of the expandable anchoring structure thereby at least partially anchoring the prosthetic implant to the native aorta. In some embodiments, the one or more backstop elements of the expandable anchoring structure are sized and configured to prevent one or more of the native leaflets from blocking flow into one or more coronary arteries by ensuring the one or more native leaflets cannot expand beyond the backstops and block one or more ostia of a right coronary artery and/or left coronary artery. In some embodiments, the expandable anchoring structure engages one or more aortic sinuses thereby anchoring the prosthetic implant and/or promoting a seal at within the region just above a sinotubular junction in the native aorta. In some embodiments, the one or more expandable anchoring structures, configured to extend within a left and a right aortic sinus, is at least partially uncovered such that the left and right coronary ostia remain uncovered by the prosthetic implant when in use. In some embodiments, the expandable anchoring structure further comprises a metallic wire frame.

In some embodiments, an expandable arch support structure and/or an expandable root support structure comprises a non-porous layer, for example, to wall off a dissection. In some embodiments, the non-porous layer is configured to expand and, upon expansion, apply a radially outward force to the ascending aorta thereby forming a seal against the internal surface of the aorta and/or anchoring the prosthetic implant to the native aorta. In some embodiments, the non-porous layer expands due to a blood hydrostatic pressure created by blood flowing through an intraluminal space formed between the non-porous layer and the porous layer. In some embodiments, the non-porous layer contacts the inner wall of the native aorta between a brachiocephalic trunk and the sinotubular junction of the native aorta. In some embodiments, the non-porous layer is configured to be positioned across at least a portion of a dissection. In some embodiments, the non-porous layer is configured to seal around at least a portion of the dissection. In some embodiments, the non-porous layer is configured to prevent blood flow through the dissection. In some embodiments, the non-porous layer comprises an opening to allow blood to flow from within the expandable support structure, through the opening, and into the carotid and the subclavian arteries of the native aorta. In some embodiments, the non-porous layer comprises a second coating. In some embodiments, the second coating prevents adhesion of at least one or more component of blood. In some embodiments, the second coating comprises a therapeutic agent.

In some embodiments, an expandable arch support structure and/or an expandable root support structure comprises a porous layer, for example, in place of the one or more expandable branches (e.g., an expandable arch support graft comprising zero, one, or two expandable branches). As one of skill in the art will appreciate, the porous layer is intended to permit blood flow from inside the expandable arch support graft and into the vessels of the aortic arch. Any suitable porous layer known to the skilled artisan may be used in the expandable arch support structures described herein. In some embodiments, the porous layer has an areal density of between 0-300 g/m\hat{ }2. In some embodiments, the porous layer has an average areal density of between 50-300 g/m\hat{ }2. In some embodiments, the second porous layer has an average 0.01-5.00 mm. In some embodiments, the porous layer has an average pore size of between 0.01-2.00 mm. In some embodiments, the second porous layer has a thickness of between 0-200 microns. In some embodiments, the porous layer has a thickness of between 10-200 microns. In some embodiments, the porous layer comprises a coating. In some embodiments, the porous layer prevents adhesion of at least one or more components of blood to the porous layer. In some embodiments, the coating further comprises a therapeutic.

The prosthetic implants of the present disclosure, may according to some embodiments, comprise any combination of structures and/or configurations disclosed herein. For example, in some embodiments, the expandable root support structure comprises a metallic frame that extends continuously from the proximal end to a distal end of the expandable root support structure and that is continuous with the metallic frame of the expandable anchoring structure. In some embodiments, the expandable root support structure and the expandable anchoring structure comprise separate frames. In some embodiments, the expandable root support structure and the expandable anchoring structure are formed from a single continuous wire.

In some embodiments, the expandable arch support structure is sized and configured to be positioned within at least a portion of the descending portion of the native aorta. In some embodiments, the expandable arch support structure is configured to permit blood flow from within the expandable arch support structure, through a porous layer, and into one or more carotid arteries and/or subclavian arteries of the native aorta. In some embodiments, the expandable arch support structure, comprising the porous layer, is configured to substantially cover the descending aorta to a brachiocephalic trunk of the native aorta and an expandable root support structure, comprising a non-porous layer, is configured to engage a wall of the ascending aorta on opposite sides of a tear of the dissection.

In some embodiments, an expandable arch support structure comprises a metallic frame that extends continuously from a proximal end to a distal end of the expandable arch support structure and that is continuous with the metallic frame of an expandable anchoring structure. In some embodiments, the expandable arch support structure and the expandable anchoring structure comprise separate frames.

In some embodiments, an expandable root support structure, an expandable anchoring structure, and an expandable arch support structure comprise separate frames. In some embodiments, the expandable root support structure, the expandable anchoring structure, and the expandable arch support structure are formed from a single continuous wire.

In some embodiments, an expandable arch support structure and an expandable anchoring structure are formed from a single continuous wire.

In some embodiments, when loaded into a delivery device the one or more expandable branches configured to be positioned within the left subclavian is in a crimped position and sits parallel to a distal portion of the expandable arch support structure.

In some embodiments, an expandable arch support structure is pre-formed with a curvature to conform to an aortic arch of the native aorta.

In some embodiments, an expandable root support structure is configured to be coupled to an expandable arch support structure, thereby anchoring the expandable root support structure to the expandable arch support structure. In some embodiments, the expandable root support structure configured to be positioned within an ascending portion of the native aorta is coupled to an expandable arch support structure configured to be positioned within at least a portion of the descending portion of the native aorta. In some embodiments, an expandable arch support structure does not apply a radially outward force against the native aorta.

The skilled artisan will understand that a prosthetic implant as disclosed herein may be used to treat any suitable disease or condition known to the skilled artisan. For example, in some embodiments, the implant is configured to treat an aortic dissection. In some cases, the aortic dissection is a Type A aortic dissection. Treatment of other conditions is also contemplated herein, according to other embodiments. For example, in some embodiments, the implant is configured to treat an intramural hematoma. Additionally, or alternatively, the implants disclosed herein may be used to treat a thoracic aortic aneurysm.

Other aspects of the disclosure generally relate to a graft delivery system. In some embodiments, the graft delivery system comprises an outer sheath, the outer sheath configured to move along a first guidewire and a second guidewire. In some embodiments, the graft delivery system further comprises an arch graft sheath inside the outer sheath, the arch graft sheath configured to move along a first guidewire, the arch graft sheath pre-loaded with an expandable arch support structure and configured to release the expandable arch support structure at least partially in a descending aorta and an ascending aorta. In some embodiments, the graft delivery system further comprises a first expandable branch of the expandable arch support structure pre-loaded in the outer sheath, the first expandable branch configured to move along the second guidewire.

In some embodiments, the arch graft sheath is separable into a first portion and a second portion. In some embodiments, the first portion comprises a descending portion of the expandable arch support structure. In some embodiments, the first portion comprises an ascending portion of the expandable arch support structure. In some embodiments, the second portion comprises a descending portion of the expandable arch support structure. In some embodiments, the second portion comprises an ascending portion of the expandable arch support structure. In some embodiments, the first expandable branch is pre-loaded in the outer sheath, the first expandable branch is in a crimped state and sits parallel to the first portion of the arch graft sheath. In some embodiments, the first expandable branch is pre-loaded in the outer sheath, the first expandable branch is in a crimped state and sits parallel to the second portion of the arch graft sheath. In some embodiments, when the first expandable branch is pre-loaded in the outer sheath, the first expandable branch is in an inverted state. In some embodiments, further comprising a nose cone on a distal end of the arch graft sheath. In some embodiments, the nose cone is removably attached to the outer sheath. In some embodiments, the outer sheath is pre-loaded with an expandable root support structure and is configured to release the expandable root support structure in the ascending aorta. In some embodiments, the first expandable branch of the expandable arch support structure comprises a stent on a distal end of the head vessel graft.

In some embodiments, the graft delivery system further comprises a nose cone on a distal end of the inner sheath. In some embodiments, the inner sheath is separable into a first portion and a second portion. In some embodiments, the outer sheath is configured to contain a second expandable branch of the expandable support structure.

Additional aspects of the disclosure relate to a prosthetic implant system. In some embodiments, the prosthetic implant system comprises an outer sheath, the outer sheath configured to move along a first guidewire and a second guidewire. In some embodiments, the prosthetic implant system comprises an inner sheath inside the outer sheath, the inner sheath configured to move along a first guidewire, the inner sheath configured to carry an expandable arch support structure. In some embodiments, the prosthetic implant system comprises a first expandable branch of the expandable arch support structure inside the outer sheath, the first expandable branch configured to move along the second guidewire.

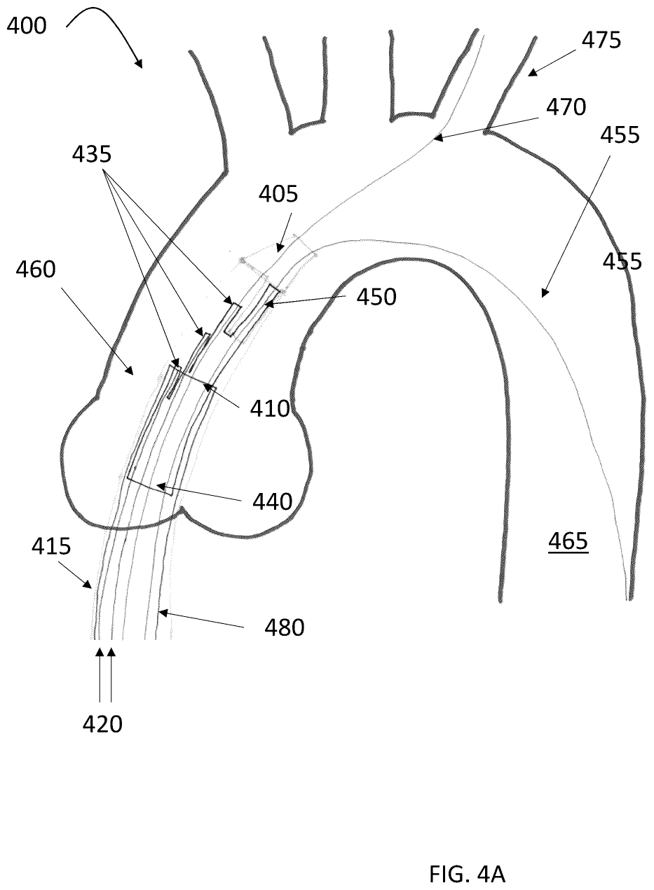

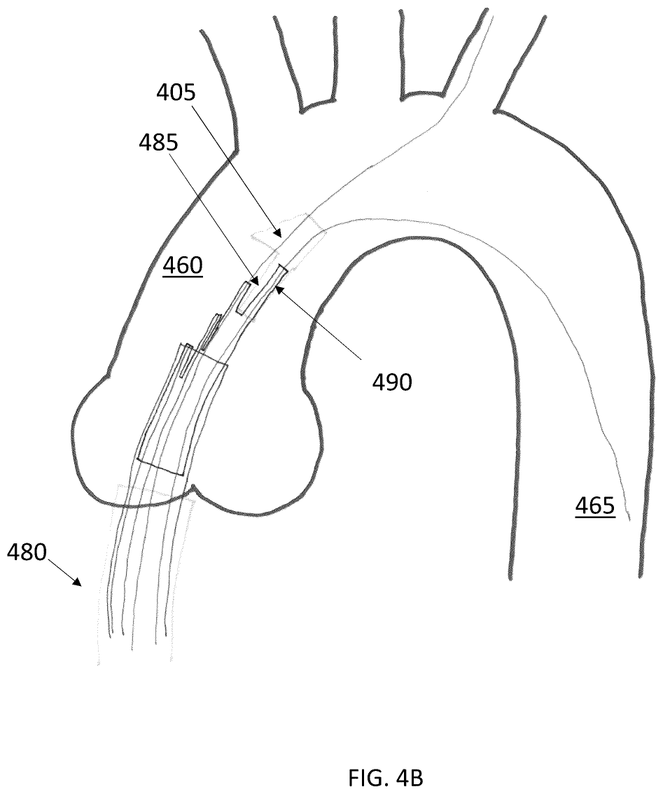

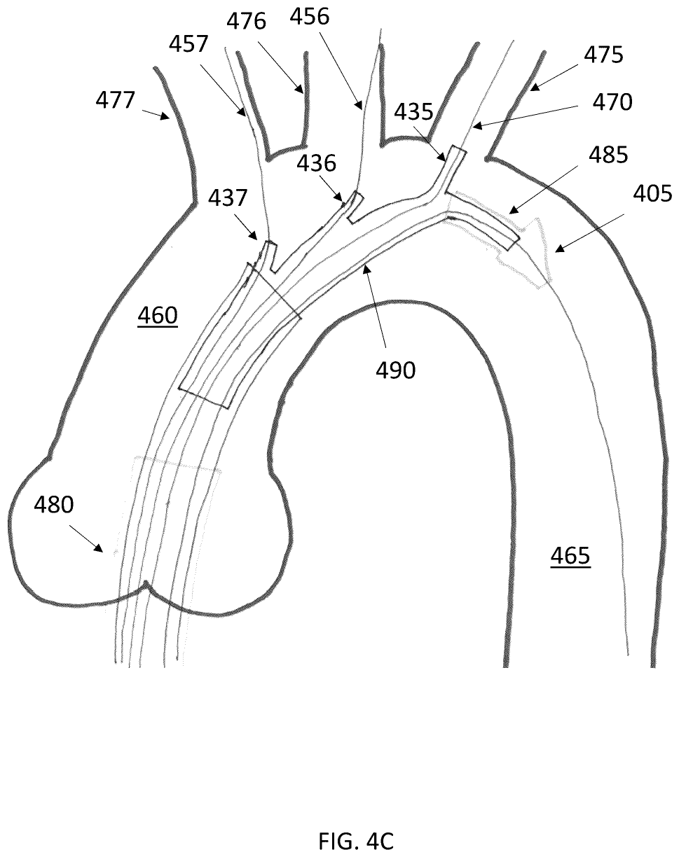

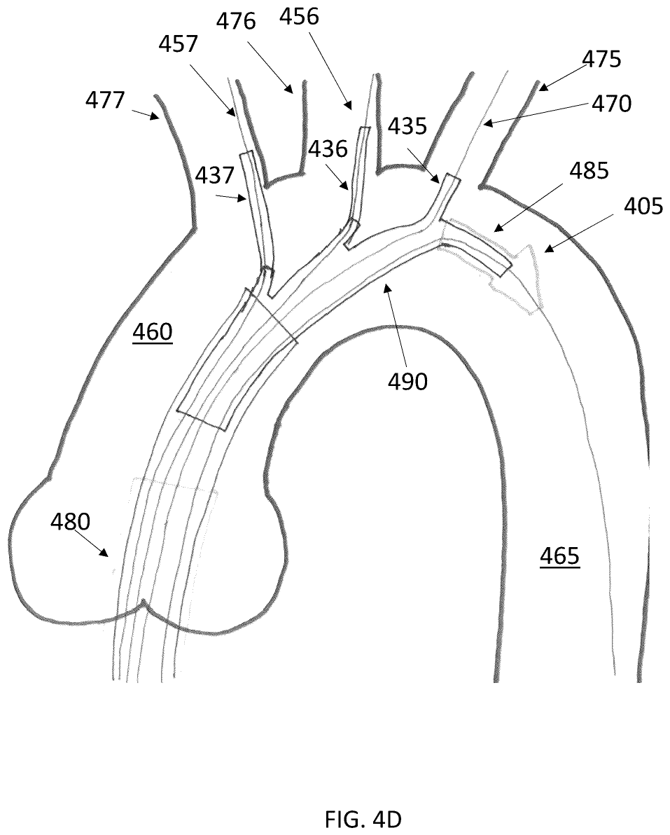

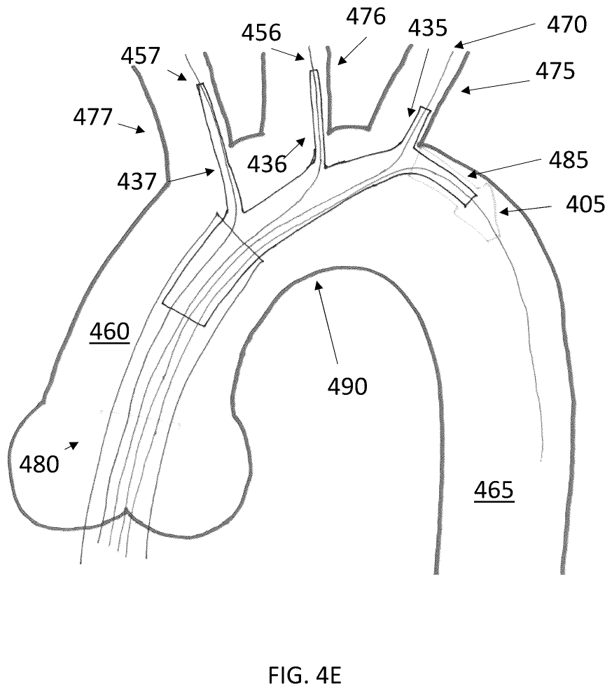

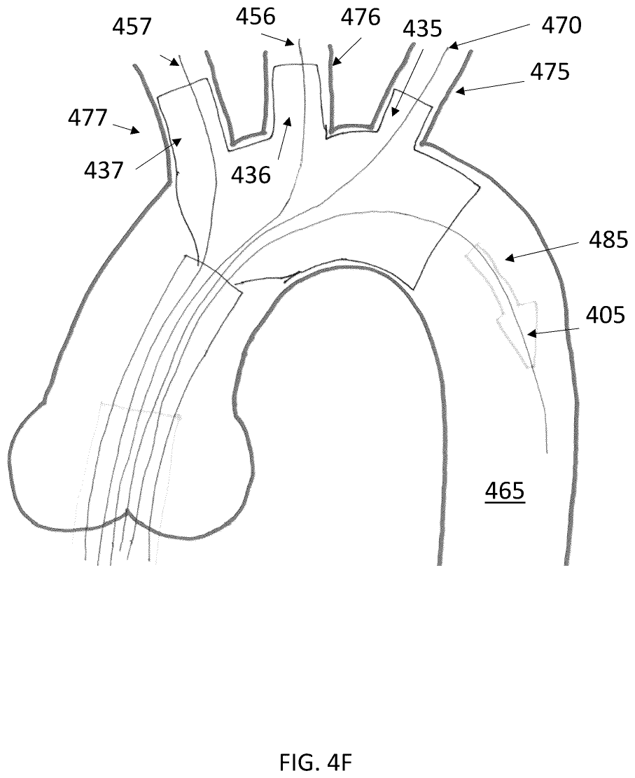

Certain aspects of the disclosure further relate to one or more methods. In some embodiments, the methods relate to treating a dissection, for example, using a prosthetic implant, graft delivery system, and/or prosthetic implant system, as disclosed herein. FIG. 4A illustrates an exemplary graft delivery system 400 comprising a nosecone 405, an ascending sheath 410, outer sheath 415, and preloaded guide wires 420. In some embodiments, exemplary graft delivery system 400 houses prosthetic implant system 430 comprising one or more expandable branches 435. In some embodiments, a proximal portion 440 of prosthetic implant system 430 is distal to comprising a nosecone 405. In some embodiments, a distal portion 450 of prosthetic implant system 430 is proximal to nosecone 405.

In some embodiments, the method is a transapical method for delivering an aortic arch implant into an aortic arch of a subject in need thereof (e.g., FIGS. 4A-4G). In some embodiments, the transapical method comprises advancing a first guidewire 455 into an ascending aorta 460 and a descending aorta 465. In some embodiments, the method further comprises advancing a second guidewire 470 into the ascending aorta 460 and a left subclavian artery 475. Additionally, in some embodiments, the methods comprise advancing a prosthetic implant delivery system 400 into the ascending aorta 460 over the first guidewire 455 and the second guidewire 470, the prosthetic implant delivery system 400 comprising an outer sheath 415, an ascending sheath, and a nosecone 405 operatively connected to descending sheath 485, the ascending sheath 480 carrying an ascending portion (e.g., proximal portion 440) of an expandable arch support structure, the descending sheath 485 carrying a descending portion (e.g., distal portion 450) of the expandable arch support structure 490, the descending sheath 485 extending through the outer sheath 415. As shown in FIG. 4B, in some embodiments, the methods further comprise retracting the outer sheath 415 in the ascending aorta 460 to expose the descending sheath 485 over the first guidewire 455. As shown in FIG. 4C, in some embodiments, the methods comprise advancing a first expandable branch 435 of the expandable arch support structure 490 from the outer sheath into the left subclavian artery 475 over the second guidewire 470, wherein the expandable branch 435 comprises a telescoping structure comprising a proximal portion, a middle portion, and a distal portion. In some embodiments, the methods comprise advancing a second expandable branch 436 of the expandable arch support structure 490 from the outer sheath 415 into the left common carotid artery 476 over a first preloaded guide wire 456, wherein second expandable branch 436 comprises a telescoping structure comprising a proximal portion, a middle portion, and a distal portion. In some embodiments, the methods comprise advancing a third expandable branch 437 of the expandable arch support structure 490 from the outer sheath 415 into a brachiocephalic artery 477 over a second preloaded guide wire 457, wherein third expandable branch 437 comprises a telescoping structure comprising a proximal portion, a middle portion, and a distal portion.

In some embodiments, the methods comprise advancing the nosecone 405 and descending sheath 485 at least partially into the descending aorta to expose distal portion 450 of expandable arch support structure 490 within at least a portion of descending aorta 465.

In some embodiments, the methods comprise retracting ascending sheath 480 expose proximal portion 440 of expandable arch support structure 490 within at least a portion of the ascending portion.

In some embodiments, the methods comprise advancing a first guidewire into a descending aorta and an ascending aorta. In some embodiments, the methods further comprise advancing a second guidewire into the descending aorta and a brachiocephalic artery. In some embodiments, the methods comprise advancing a prosthetic implant system into a descending aorta over the first guidewire and the second guidewire, the prosthetic implant delivery system comprising an outer sheath, an ascending sheath, and a descending sheath, the ascending sheath carrying an ascending portion of an expandable arch support structure, the descending sheath carrying a descending portion of the expandable arch support structure, the descending sheath extending through the outer sheath. In some embodiments, the methods further comprise retracting an outer sheath in the descending aorta to expose the ascending sheath over the first guidewire and advancing a first expandable branch of the second expandable support structure from the outer sheath into the brachiocephalic artery over the second guidewire. In some embodiments, the expandable branch comprises a telescoping structure comprising a proximal portion, a middle portion, and a distal portion. In some embodiments, the methods comprise exposing the descending portion of the expandable arch support structure from the descending sheath.

In some embodiments, the methods comprise advancing a first guidewire into an aorta and advancing a second guidewire into the aorta and into at least a portion of a first vessel of an aortic arch. In some embodiments, the methods further comprise advancing a prosthetic implant system into the aorta over the first guidewire and the second guidewire, the prosthetic implant system comprising an outer sheath and an inner sheath extending through the outer sheath, the inner sheath carrying a second expandable support structure. In some embodiments, the methods comprise retracting the outer sheath in the aorta to expose the inner sheath over the first guidewire and advancing a first expandable branch of the expandable arch support structure from the outer sheath into the first head vessel over the second guidewire. In some embodiments, the expandable branch comprises a telescoping structure comprising a proximal portion, a middle portion, and a distal portion. In some embodiments, the methods comprise exposing the second expandable support structure portion from the inner sheath in the aorta.

In some embodiments, a prosthetic implant, graft delivery system, and/or prosthetic implant system, as disclosed herein, is used for treating an aortic dissection and is delivered transapically. In some embodiments, when advancing the prosthetic implant system into the aorta, the first expandable branch of the expandable arch support structure is in a crimped state and sits parallel to the descending sheath of the prosthetic implant system. In some embodiments, advancing the first expandable branch of the expandable arch support structure comprises extending the first expandable branch away from the descending sheath of the prosthetic implant system and into the left subclavian artery. In some embodiments, the methods further comprising advancing a third guidewire into at least one of the left common carotid artery or the brachiocephalic artery. Additionally, the methods may further comprise advancing a fourth guidewire into at least one of the left common carotid artery or the brachiocephalic artery.

In some embodiments, the methods further comprising advancing a second expandable branch of the expandable arch support structure into a left common carotid artery, optionally, at least partially retracting the ascending sheath to expose at least a portion of the second expandable branch prior to advancing the second expandable branch. In some embodiments, the second expandable branch is at least partially inverted within the prosthetic implant system prior to advancing the second expandable branch of the expandable arch support structure. In some embodiments, the methods comprise releasing the second expandable branch from the inverted state prior to advancing the second expandable branch of the expandable arch support structure into left common carotid artery.

In some embodiments, the methods further comprise advancing a third expandable branch of the expandable arch support structure into a brachiocephalic artery, optionally, at least partially retracting the ascending sheath to expose at least a portion of the third expandable branch prior to advancing the third expandable branch. In some embodiments, the third expandable branch is at least partially inverted within the prosthetic implant system prior to advancing the third expandable branch of the expandable arch support structure. In some embodiments, the methods comprise releasing the third expandable branch from the inverted state prior to advancing the third expandable branch of the expandable arch support structure into the brachiocephalic artery.

In some embodiments, the methods comprise inflating a balloon to expand at least one of the expandable branches of the expandable arch support structure following placement of the expandable branch within the left subclavian artery, left common carotid artery, and/or the brachiocephalic artery.

In some embodiments, the methods comprise advancing the descending sheath distally to deploy the descending portion of the expandable arch support structure. In some embodiments, the methods comprise retracting the ascending sheath proximally to deploy the ascending portion of the expandable arch support structure. In some embodiments, the methods further comprise advancing the first guidewire or the second guidewire into at least one of the left common carotid artery or the brachiocephalic artery. In some embodiments, the prosthetic implant delivery system is advanced transapically over the first guidewire and the second guidewire.

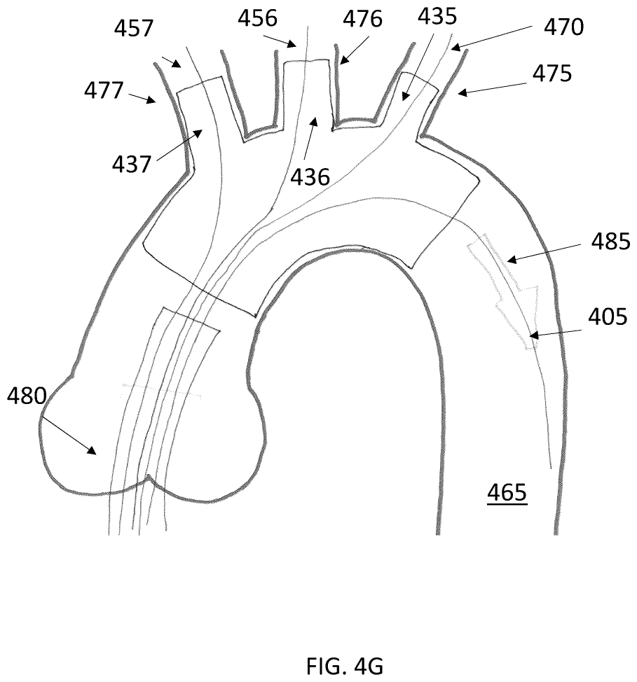

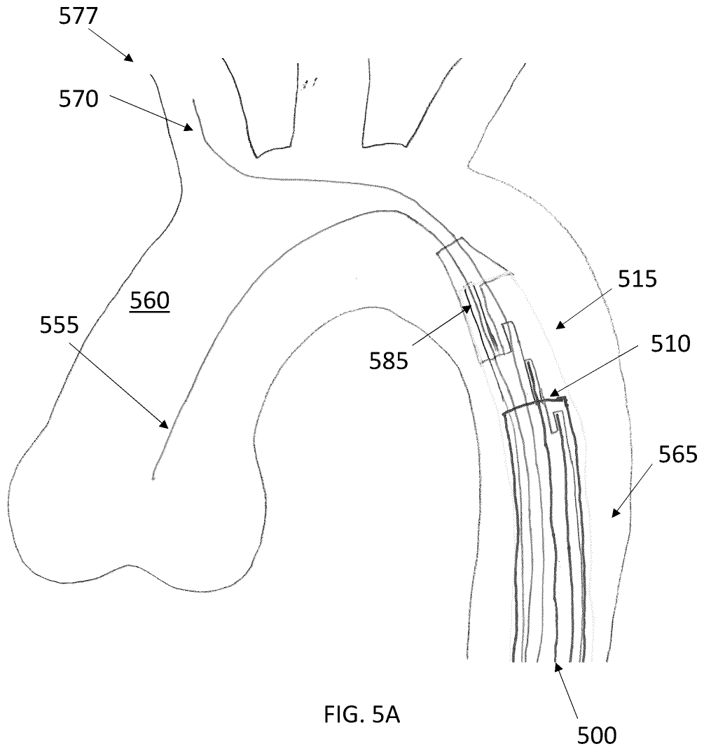

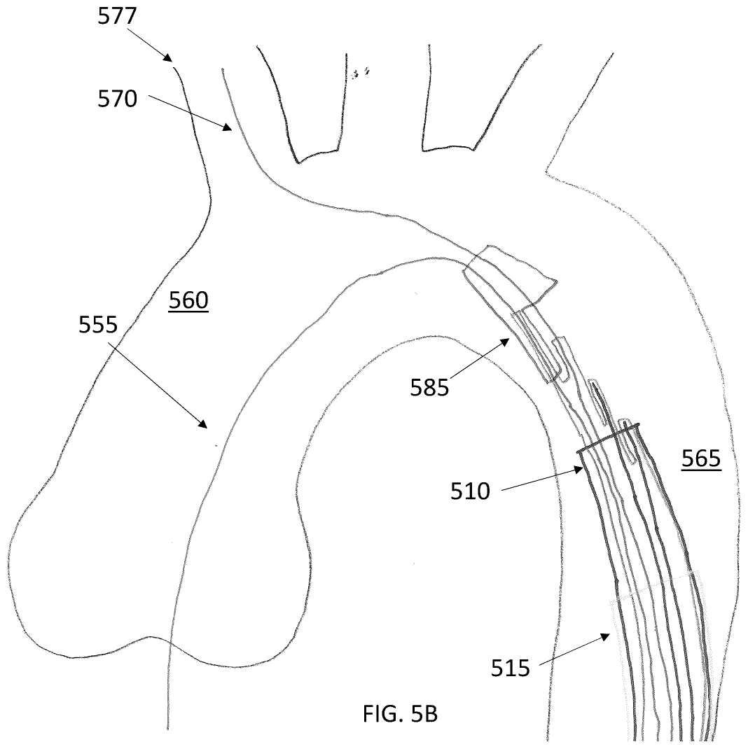

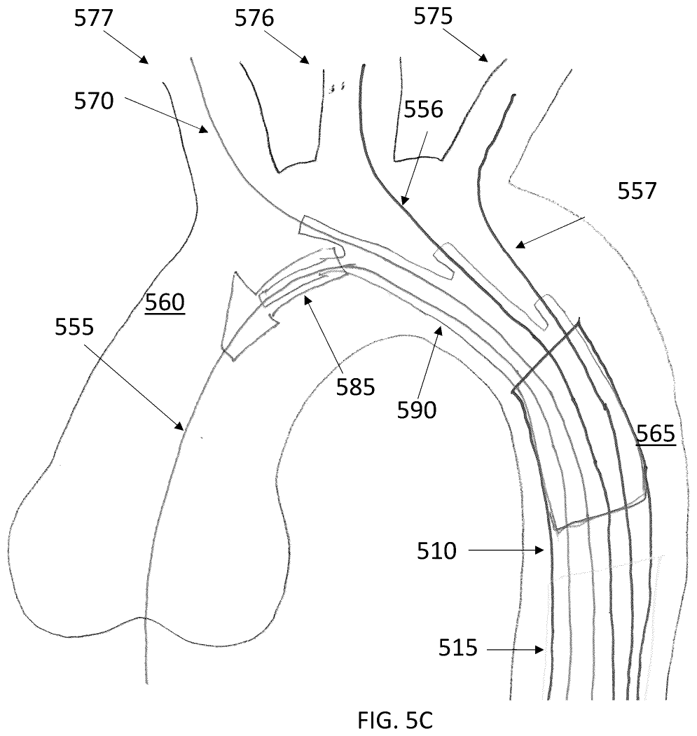

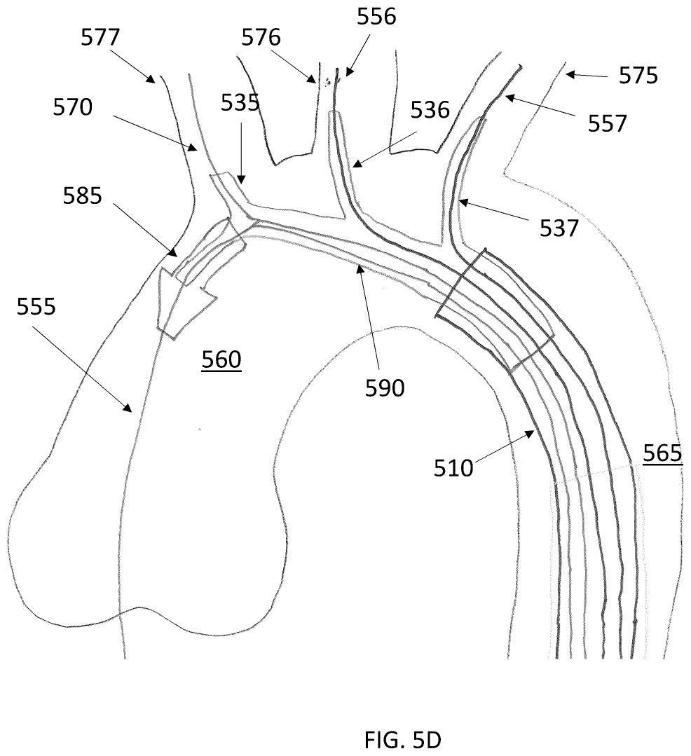

In some embodiments, the method is a transfemoral method for delivering an aortic arch implant into an aortic arch of a subject in need thereof (e.g., FIGS. 5A-5E). In some embodiments, the transfemoral method comprises advancing a first guidewire 555 into descending aorta 565 and into an ascending aorta 560. In some embodiments, the method further comprises advancing a second guidewire 570 into the descending aorta 565 and into a brachiocephalic artery 577. Additionally, in some embodiments, the methods comprise advancing a prosthetic implant delivery system 500 into the descending aorta 565 over the first guidewire 555 and the second guidewire 570, the prosthetic implant delivery system 500 comprising an outer sheath 515, a descending sheath 510, and a nosecone 505 operatively connected to ascending sheath 585, the ascending sheath 585 carrying an ascending portion (e.g., proximal portion 540) of an expandable arch support structure, the descending sheath 510 carrying a descending portion (e.g., distal portion 550) of the expandable arch support structure 590, the ascending sheath 585 extending through the outer sheath 515. As shown in FIG. 5B, in some embodiments, the methods further comprise retracting the outer sheath 515 in the descending aorta 565 to expose the descending sheath 510 over the first guidewire 555. As shown in FIGS. 5C and 5D, in some embodiments, the methods comprise advancing a first expandable branch 535 of the expandable arch support structure 590 from the outer sheath 515 into a brachiocephalic artery 577 over the second guidewire 570, wherein the expandable branch 535 comprises a telescoping structure comprising a proximal portion, a middle portion, and a distal portion. In some embodiments, the methods comprise advancing a second expandable branch 536 of the expandable arch support structure 590 from the outer sheath 515 into the left common carotid artery 576 over a first preloaded guide wire 556, wherein second expandable branch 536 comprises a telescoping structure comprising a proximal portion, a middle portion, and a distal portion. In some embodiments, the methods comprise advancing a third expandable branch 537 of the expandable arch support structure 590 from the outer sheath 515 into a left subclavian artery 575 over a second preloaded guide wire 557, wherein third expandable branch 537 comprises a telescoping structure comprising a proximal portion, a middle portion, and a distal portion.

FIG. 5E illustrates, according to some embodiments, advancing the nosecone 505 and ascending sheath 585 at least partially into the ascending aorta 560 to expose proximal portion 540 of expandable arch support structure 590 within at least a portion of the ascending aorta 560.

In some embodiments, the methods comprise retracting descending sheath 510 into the descending aorta 565 to expose a distal portion 550 of expandable arch support structure 590 within at least a portion of the descending aorta 565.

In some embodiments, first expandable branch 535, second expandable branch 536, and/or third expandable branch 537 each comprise a distal expandable frame configured to expand upon placement within one of the great vessels of the aortic arch (e.g., left subclavian artery, left common carotid artery, or brachiocephalic artery).

In some embodiments, a prosthetic implant, graft delivery system, and/or prosthetic implant system, as disclosed herein, is used for treating an aortic dissection and is delivered transfemorally. For example, in some embodiments, when advancing the prosthetic aortic implant system (or graft delivery system or prosthetic implant) into the aorta, the first expandable branch of the expandable arch support structure is in a crimped state and sits parallel to the ascending sheath of the prosthetic implant system. In some embodiments, advancing the first expandable branch of the expandable arch support structure comprises extending the first expandable branch away from the ascending sheath of the prosthetic implant system and into the brachiocephalic artery. In some embodiments, the methods comprise advancing a third guidewire into at least one of the left common carotid artery or the left subclavian artery. In some embodiments, the methods further comprise advancing a fourth guidewire into at least one of the left common carotid artery or the left subclavian artery.

The method, according to some embodiments, further comprises advancing a second expandable branch of the expandable arch support structure into a left common carotid artery, optionally, at least partially retracting the descending sheath to expose at least a portion of the second expandable branch prior to advancing the second expandable branch. In some embodiments, prior to advancing the second expandable branch of the expandable arch support structure, the second expandable branch is at least partially inverted within the prosthetic implant system. In some embodiments, advancing the second expandable branch of the expandable arch support structure comprises releasing the second expandable branch from the inverted state and into left common carotid artery.

In some embodiments, the methods further comprise advancing a third expandable branch of the expandable arch support structure into a left subclavian artery, optionally, at least partially retracting the descending sheath to expose at least a portion of the third expandable branch prior to advancing the third expandable branch. In some embodiments, prior to advancing the third expandable branch of the expandable arch support structure, the third expandable branch is at least partially inverted within the prosthetic implant system. In some embodiments, advancing the third expandable branch of the expandable arch support structure comprises releasing the third expandable branch from the inverted state and into the brachiocephalic artery.

In some embodiments, the methods further comprise advancing the ascending sheath to deploy the ascending portion of the expandable arch support structure. In some embodiments, the methods comprise retracting the descending sheath to deploy the descending portion of the expandable arch support structure. In some embodiments, the methods comprise advancing the first guidewire or the second guidewire into at least one of the left common carotid artery or the left subclavian artery. In some embodiments, prosthetic implant delivery system is advanced transfemorally over the first guidewire and the second guidewire.

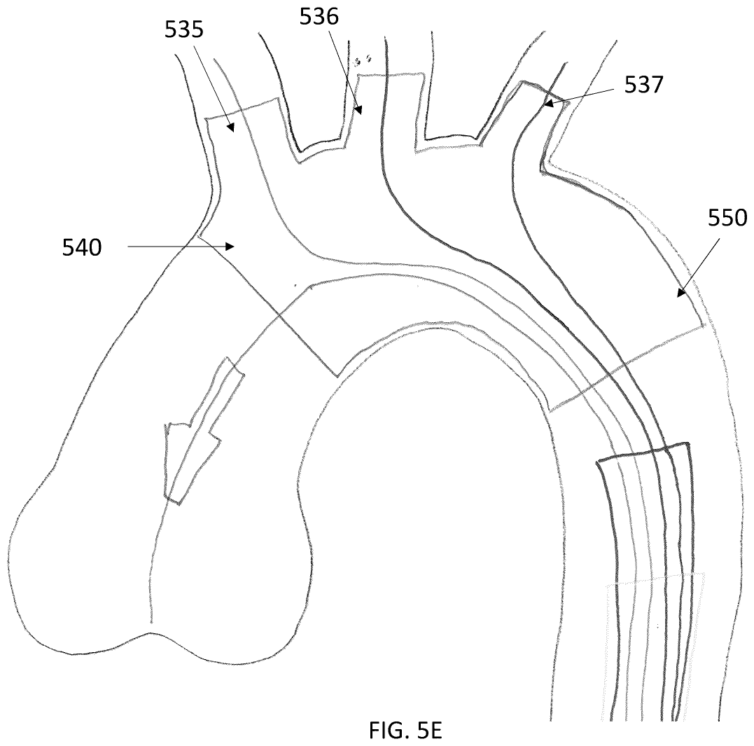

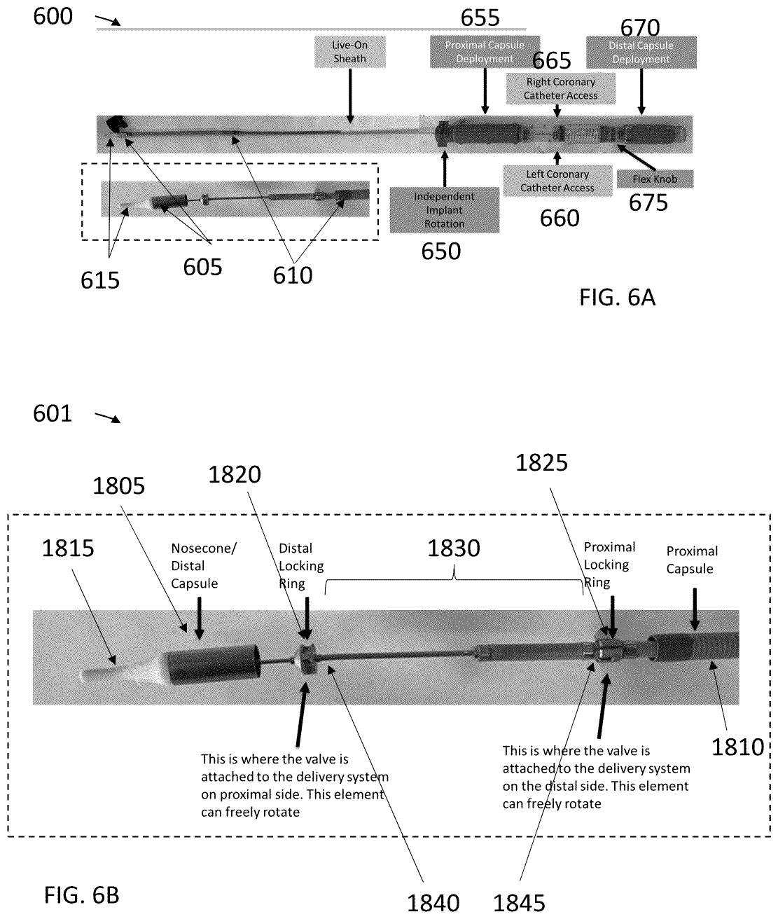

Other aspects of the disclosure relate to a graft delivery systems configured to enable rotation of the implant within the delivery device. FIG. 6A shows an intact exemplary prosthetic implant delivery system 600 comprising nosecone 615, distal capsule 605, proximal capsule 610, independent implant rotation control 650, proximal capsule deployment control 655, left coronary catheter access control 660, right coronary catheter access control 665, distal capsule deployment control 670, and flex knob 675.

FIG. 6B shows an exemplary prosthetic implant delivery system 601, wherein the distal capsule 605 has been separated from the proximal capsule 610 to reveal the internal components of the system. In some embodiments, the prosthetic implant delivery system 601 comprises a distal capsule 605 (e.g., comprising a nosecone 615), a distal locking ring 620, a proximal locking ring 625, a proximal capsule 610, and a prosthetic implant 630. In some embodiments, the distal locking ring 620 is configured to engage a distal end 640 of the prosthetic implant 630 (e.g., end comprising the artificial valve, useful for transfemoral delivery). Similarly, in some embodiments, the proximal locking ring 625 is configured to engage a proximal end 645 of the prosthetic implant (e.g., end comprising the artificial valve, useful for transapical delivery).

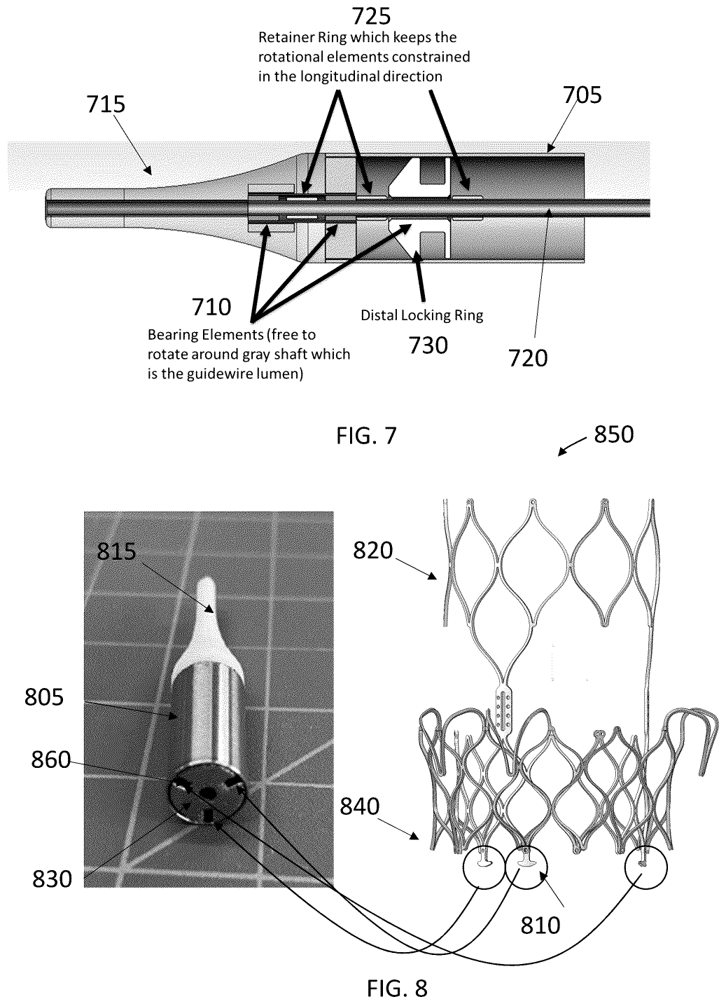

FIG. 7 shows a computer automated drawing illustrating an exemplary assembly of the nosecone and the distal locking ring shown in FIG. 6. As illustratively shown in FIG. 7, in some embodiments, the nosecone 715 and distal capsule 705 comprises a plurality of bearing elements 710 that freely rotate around a guide wire lumen 720. The nosecone 715 and distal capsule 705 further comprise a plurality of retainer rings 725 that keep the rotational elements constrained in the longitudinal direction. Additionally, as shown illustratively in FIG. 7, the device also comprises a distal locking ring 730 that is also flanked on both sides by a plurality of retainer rings 725. The distal locking ring 730 also freely rotates around the guide wire lumen 720. While not shown explicitly in FIG. 7, a proximal ring may also be flanked on both sides by a plurality of retainer rings 725 and can also freely rotate around the central elongate shaft.

The skilled artisan will understand that for transapical delivery methods, the proximal locking ring is configured to engage the portion of the expandable root support structure comprising an artificial valve. Likewise, for transfemoral delivery methods, the distal locking ring is configured to engage the portion of the expandable root support structure comprising the artificial valve.

The skilled artisan will understand that the orientation of the prosthetic implant between the distal and proximal locking rings is non-limiting. Thus, in some embodiments, a proximal end of the prosthetic implant may be engaged with the distal locking ring. In some embodiments, in some embodiments, a proximal end of the prosthetic implant may be engaged with the proximal locking ring. Likewise, in some embodiments, a distal end of the prosthetic implant may be engaged with the distal locking ring. Alternatively, in some embodiments, the distal end of the prosthetic implant may be engaged with the proximal locking ring.

In some embodiments a distal end of the expandable root support structure is operatively connected to the distal locking ring of the graft delivery system and a proximal end of the expandable root support structure is connected to the proximal locking ring. FIG. 8 illustrates an exemplary connection between the distal locking ring 830 and a plurality of connectors 810 at a distal end 840 of inner frame 820 of expandable root support structure 850. As shown illustratively in FIG. 8, in some embodiments, the distal locking ring 830 comprises a plurality of openings 860. In some embodiments, the plurality of openings 860 of the distal locking ring 830 are configured to receive a plurality of connectors 810 at a distal end 840 of inner frame 820 of expandable root support structure 850. The connectors 810 may have any suitable shape for attaching, mating, and/or being inserting into the openings 860 of the distal locking ring 830. Likewise, in some embodiments, a proximal locking ring comprises a second plurality of openings and a proximal end of the implant (e.g., expandable root support structure) comprises a second plurality of connectors. In some embodiments, the second plurality of openings of the proximal locking ring is configured to receive the second plurality of connectors of the proximal end of the implant. Other configurations are also possible.

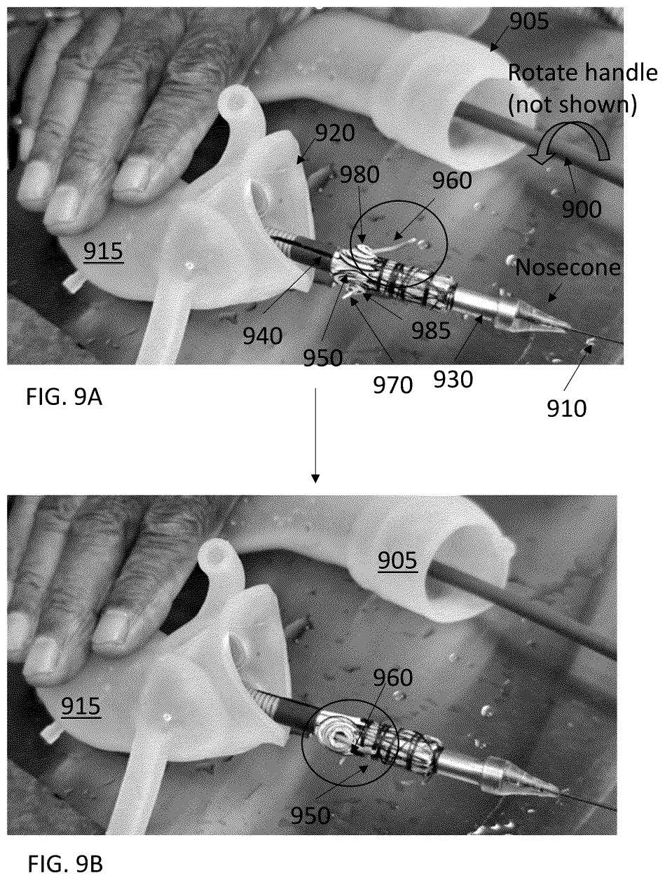

Those of skill in the art will understand that such configurations permit independent rotation of the prosthetic implant within the delivery system (e.g., the prosthetic implant can be rotated without rotating the delivery system). The inventors have discovered within the context herein that this rotatability may be useful for aligning an expandable branch of the expandable support structure with the coronary arteries. This independent rotation feature is shown illustratively in FIG. 9A and 9B. FIG. 9A shows a prosthetic implant delivery system being administered transfemorally using a cast of a human aorta. As shown in FIG. 9A, prosthetic implant delivery system 900 is guided along a first guide wire 910 up through descending aorta 905 through the ascending aorta 915 and out the aortic root 920. Distal capsule 930 is advanced out of the aortic root 920 and proximal capsule 940 is retracted toward the descending aorta 905, thus exposing a portion of expandable root support structure 950. In some embodiments, a first coronary catheter 960 and a second coronary catheter 970 pass through a first opening 980 and a second opening 985, respectively, in expandable root support structure 950. In some embodiments, rotation of a handle (not shown in FIG. 9A) permits independent rotation of the loaded prosthetic implant (e.g., expandable root support structure 950) without rotating the prosthetic implant delivery system 900. This is clearly shown in FIG. 9B, wherein first coronary catheter 960 has been rotated approximately 180 degrees.

Additionally, any one of the methods disclosed herein may further comprise any one of embodiments as disclosed herein. For example, in some embodiments, when advancing a prosthetic aortic implant system into the aorta, a first expandable branch of an expandable arch support structure is in a crimped state and sits parallel to a descending portion of the prosthetic implant system. In some embodiments, wherein advancing the first expandable branch of the expandable arch support structure comprises extending the first expandable branch away from the descending portion of the prosthetic implant system and into the first vessel of the aortic arch. In some embodiments, the first vessel of the aortic arch is the left subclavian artery.

In some embodiments, the methods comprise advancing a second expandable branch of an expandable arch support structure into a second vessel of the aortic arch using, for example, a third guidewire. Prior to advancing, the second expandable branch of the expandable arch support structure is at least partially inverted within the prosthetic implant system, according to some embodiments. Additionally, in some embodiments, advancing the second expandable branch of the expandable arch support structure comprises releasing the second expandable branch from the inverted state and into the second vessel of the aortic arch. In some embodiments, the second vessel of the aortic arch is the left common carotid artery.

In some embodiments, the methods further comprise advancing a third expandable branch of the expandable arch support structure into a third vessel of the aortic arch using a fourth guidewire. In some embodiments, prior to advancing, the third expandable branch of the expandable arch support structure is at least partially inverted within the prosthetic implant system. In some embodiments, advancing the third expandable branch of the expandable arch support structure comprises releasing the third expandable branch from the inverted state and into the third vessel of the aortic arch. In some embodiments, the third vessel of the aortic arch is the brachiocephalic artery.

In some embodiments, the methods further comprise inflating a balloon to expand at least one of the expandable branches of the expandable arch support structure. In some embodiments, the methods further comprise exposing at least one of the expandable branches of the expandable arch support structure from a sheath.

In some embodiments, prosthetic implant system is advanced transapically over the first guidewire and the second guidewire. In other embodiments, the prosthetic implant system is advanced transfemorally over the first guidewire and the second guidewire.

In some embodiments, a graft delivery system does not comprise rotatable elements (e.g., bearing elements, retainer rings, distal locking ring, or proximal locking ring). For example, in some embodiments, the graft delivery system comprises an outer sheath, the outer sheath configured to move along a first guidewire. In some embodiments, the system further comprises a distal sheath extending through the outer sheath, the outer sheath carrying an expandable root support structure. In some embodiments, the system comprises a first expandable branch of the expandable support structure inside the outer sheath, the first expandable branch configured to move along a first coronary access wire.

### Equivalents and Scope

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A prosthetic implant, comprising:
an expandable arch support structure comprising one or more openings and one or more expandable branches configured to be positioned within the one or more openings, wherein the expandable arch support structure is configured to be positioned within at least a portion of a descending portion of a native aorta,
wherein the expandable arch support structure comprises:
(i) a nonporous layer and is configured to contact an outer wall of the native aorta, and
(ii) one or more changes in a cross-sectional dimension within the body of the expandable support structure
wherein the one or more expandable branches comprises a telescoping structure comprising a proximal portion, a middle portion, and a distal portion,
wherein the one or more expandable branches is configured to be positioned within at least a portion of one or more vessels of an aortic arch of the native aorta and to permit blood flow from within the expandable arch support structure into at least one or more vessels of the aortic arch of the native aorta.

2-3. (canceled)

2. A prosthetic implant, comprising:
an expandable arch support structure, comprising one or more openings and one or more expandable branches configured to be positioned within the one or more openings, sized and configured to be positioned within a descending portion of a native aorta,
an expandable root support structure, comprising one or more openings and one or more expandable branches configured to be positioned within the one or more openings, sized and configured to be positioned within an ascending portion of a native aorta, and
wherein a distal end of the expandable root support structure is configured to engage a proximal end of the expandable arch support structure,
wherein the expandable arch support structure comprises:
(i) a second non-porous layer and is configured to contact an outer wall of at least a portion of the native aorta, and
(ii) one or more expandable branches comprising a telescoping structure,
wherein the one or more expandable branches comprise a proximal portion, a middle portion, and a distal portion, and wherein the one or more expandable branches is configured to be positioned within at least a portion of one or more vessels of an aortic arch of the native aorta and to permit blood flow from within the expandable arch support structure into one or more vessels of the aortic arch of the native aorta.

3. (canceled)

4. The prosthetic implant as in any claim 1, wherein the proximal portion of the one or more expandable branches comprises ePTFE.

5. The prosthetic implant as in claim 1, wherein the middle portion and/or the distal portion of the one or more expandable branches comprises an expandable support structure.

6. The prosthetic implant as in claim 1, wherein the expandable support structure is at least partially covered with ePTFE.

7-8. (canceled)

9. The prosthetic implant as in claim 1, wherein, when the middle portion and the distal portion of the one or more expandable branches are in a crimped state, the middle and distal portions are configured to collapse into the proximal portion of the one or more expandable branches and/or lumen of the expandable arch support structure.

10. (canceled)

11. The prosthetic implant as in claim 1, wherein the middle portion of the one or more expandable branches comprises a metallic wire frame, optionally wherein the metallic wire frame comprises an ePTFE covering.

12-17. (canceled)

18. The prosthetic implant as in claim 11, wherein, when the metallic wire frame of the middle portion is in a collapsed state the middle portion is configured to sit within the proximal portion of the one or more expandable branches.

19. The prosthetic implant as in claim 1, wherein, when the expandable support structure of the distal portion is in a crimped state and the middle portion is in a collapsed state, the distal and middle portions are configured to fit within the proximal portion and/or within a lumen of the expandable support structure.

20. The prosthetic implant as in claim 1, wherein the proximal portion of the one or more expandable branches is configured to be coupled to the middle portion of the one or more expandable branches and the middle portion of the one or more expandable branches is configured to be coupled to the distal portion of the one or more expandable branches.

21. The prosthetic implant as in claim 1, wherein the one or more expandable branches is configured to pivot radially between 0 degrees and 360 degrees, relative to a first axis.

22. The prosthetic implant as in claim 1, wherein the one or more expandable branches is configured to pivot laterally between 90 degrees and −90 degrees, relative to a second axis.

23. The prosthetic implant as in claim 1, wherein the one or more expandable branches is configured to be in an extended state.

24. The prosthetic implant as in claim 1, wherein the one or more expandable branches is configured to be in a collapsed state.

25-27. (canceled)

28. The prosthetic implant as in claim 1, wherein, in an expanded state, an inner diameter of the proximal portion of the one or more expandable branches is approximately the same as an inner diameter of the distal portion of the one or more expandable branches.

29. (canceled)

30. The prosthetic implant as in claim 1, wherein the nonporous layer comprises ePTFE.

31-32. (canceled)

33. The prosthetic implant as in claim 1, wherein the one or more expandable branches permits blood flow from within the expandable arch support structure into at least a portion of the left subclavian artery, a portion of the left common carotid artery, and/or a portion of the brachiocephalic artery of the native aorta.

34-94. (canceled)

95. The prosthetic implant as in claim 1, wherein the expandable arch support structure is sized and configured to be positioned within at least a portion of the ascending and/or descending portion of the native aorta.

96-102. (canceled)

103. The prosthetic implant as in claim 1, wherein, when loaded into a delivery device the one or more expandable branches configured to be positioned within the left subclavian is in a crimped position and sits parallel to a distal portion of the expandable arch support structure.

104. The prosthetic implant as in claim 1, wherein the expandable arch support structure is pre-formed with a curvature to conform to an aortic arch of the native aorta.

105. (canceled)

106. The prosthetic implant as in claim 1, wherein the expandable arch support structure does not apply a radially outward force against the native aorta.

107-166. (canceled)
